(19) United States
(12) Patent Application Publication
(10) Pub. No.: US 2024/0300904 A1
(43) Pub. Date: Sep. 12, 2024

(54) PYRIDAZINONES FOR THE TREATMENT OR PREVENTION OF HYPERTENSION

(71) Applicant: UNIVERSITETET I OSLO, Oslo (NO)

(72) Inventors: Lise Román MOLZAU, Nittedal (NO); Henriette ANDRESEN, Oslo (NO); Finn Olav LEVY, Oslo (NO); Jo KLAVENESS, Oslo (NO); Zakieh IZAKIAN, Bergen (NO); Alessandro CATALIOTTI, Høvik (NO); Anita WEGERT, Aldenhoven (DE); Ruben LEENDERS, Nijmegen (NL); Piotr NIECZYPOR, Nijmegen (NL)

(21) Appl. No.: 18/569,056

(22) PCT Filed: Jun. 10, 2022
(86) PCT No.: PCT/GB2022/051469

§ 371 Date: Dec. 11, 2023

(30) Foreign Application Priority Data
Jun. 10, 2021 (GB) 2108303.5

(51) Int. Cl. C07D 237/14; A61K 9/00; A61K 9/48; A61K 31/50; A61K 31/501; A61K 47/40

(52) U.S. Cl. CPC C07D 237/14; A61K 9/0019; A61K 9/4825; A61K 9/4858; A61K 9/4866; A61K 31/50; A61K 31/501; A61K 47/40

(57) ABSTRACT

The invention provides compounds of formula (I), stereoisomers, and pharmaceutically acceptable salts thereof for use in the treatment or prevention of hypertension: wherein: R1 and R2 are independently selected from H, halogen, C₁₋₆ alkyl, C₁₋₆ haloalkyl, and —CN, provided that at least one of R¹ and R² is other than H; and R³ is a group of the formula: wherein: n is 0 or 1; R⁴ is H or C₁₋₃ alkyl; L₁ is a linking group selected from a bond, —(CH₂)P— in which p is an integer from 1 to 3, and C₃₋₆ cycloalkylene; and X is selected from: (i) an aryl group optionally substituted by one or more substituents selected from C₁₋₃ alkyl, C₁₋₃ alkoxy, C₁₋₃ haloalkyl, halogen, and —CN; (ii) a 5 or 6-membered heterocyclic ring optionally substituted by one or more substituents selected from C₁₋₃ alkyl, C₁₋₃ alkoxy, C₁₋₃haloalkyl, halogen, and optionally substituted phenyl; and (iii) a cycloalkyl group optionally substituted by one or more substituents selected from C₁₋₃ alkyl, C₁₋₃ haloalkyl, and halogen.

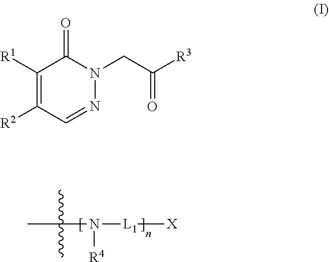

### FIELD OF THE INVENTION

The present invention relates to the use of pyridazinone derivatives in the treatment or prevention of conditions which are mediated by the natriuretic peptide receptor-A (NPR-A). In particular, it relates to the use of such compounds in the treatment or prevention of hypertension, especially resistant hypertension. The invention further relates to certain novel pyridazinone derivatives, to pharmaceutical compositions containing them, and to their use in such treatment.

### BACKGROUND OF THE INVENTION

In 2015, the World Health Organization (WHO) reported that 1.13 billion patients worldwide were diagnosed with hypertension, also known as “high blood pressure” (Cai et al., Hypertension. 2017; 70: 5-9). Hypertension is a medical condition in which the blood pressure in the arteries is persistently elevated. Long-term high blood pressure is a major risk factor for conditions such as coronary artery disease, stroke, heart failure, atrial fibrillation, peripheral arterial disease, chronic kidney disease, dementia and loss of vision. Blood pressure is expressed by two measurements, the systolic and diastolic pressures. The first (systolic) number represents the pressure in blood vessels when the heart contracts or beats. The second (diastolic) number represents the pressure in the vessels when the heart relaxes between beats. In adults, hypertension is considered to be present if the resting blood pressure is persistently at or above 140/90 mmHg.

Standard medications for hypertension are effective in large groups of patients. Current treatments involve the control of blood pressure either by monotherapy with one class of an anti-hypertensive drug, or by combination therapy with different classes of anti-hypertensive drugs. The different drug classes used in the treatment of hypertension have different mechanisms of action and include inhibitors of the renin-angiotensin-aldosterone system, thiazide diuretics, calcium channel blockers and beta-blockers. Within each class there are several drugs on the market which differ in selectivity, potency, efficacy and adverse effect profile.

There is strong evidence that anti-hypertensive drugs in combination with lifestyle changes not only reduce blood pressure, but also reduce the risk of cardiovascular disease and death. However, there is an increasing group of hypertensive patients that cannot be treated with currently available medications. These patients suffer from what is known as “resistant hypertension” (“RHT”) and are at high risk of cardiovascular morbidity and death. RHT is defined as high blood pressure that remains above a target level despite being prescribed three or more anti-hypertensive drugs simultaneously with different mechanisms of action. This group of patients represents 10-30% of the population with hypertension (Cai et al., Hypertension. 2017; 70: 5-9).

Thus there remains a need for the development of new drugs that can control elevated blood pressure, particularly in patients suffering from resistant hypertension. In particular, there is a need for new drugs for such treatment which have a different mechanism of action and which may therefore be used in combination with currently available anti-hypertensive therapies.

### SUMMARY OF THE INVENTION

The inventors now propose the treatment of hypertension and, in particular, resistant hypertension through targeting the natriuretic peptide receptor-A (“NPR-A”) using drugs which function as allosteric enhancers.

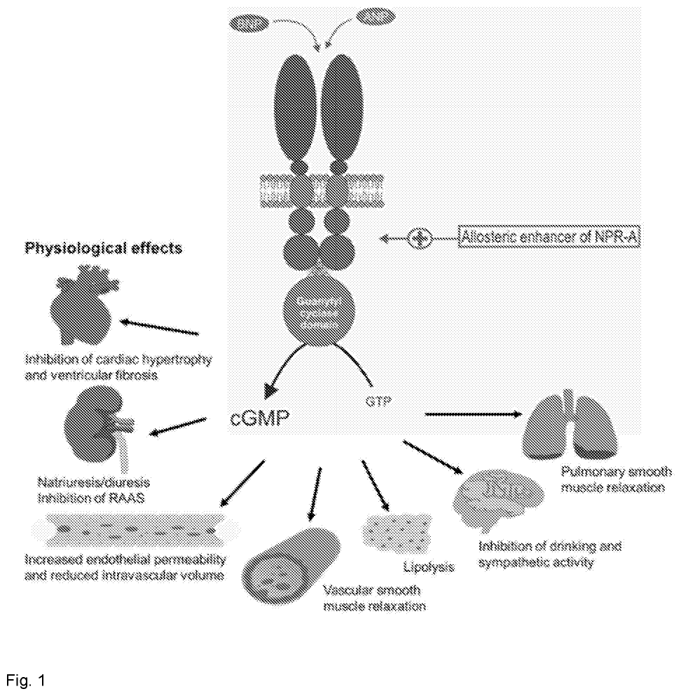

Natriuretic peptides are important in the regulation of blood pressure. Both atrial and brain natriuretic peptides (ANP and BNP, respectively) activate the receptor NPR-A, causing production of the second messenger molecule, cyclic guanosine monophosphate (cGMP), which through several mechanisms reduces blood pressure (see FIG. 1). It has been shown in animal studies that the knock-out of either ANP or BNP or the receptor itself (NPR-A) causes hypertension (John et al., Science. 1995; 267: 679-81; John et al. Am J Physiol. 1996; 271: R109-14; Holditch et al. Hypertension. 2015; 66: 199-210; Lopez et al., Nature. 1995; 378: 65-8; and Oliver et al. Proc Natl Acad Sci USA. 1997; 94: 14730-5). It has also been shown that a mutation in the NPR-A receptor is associated with increased blood pressure (Nakayama et al. Circ Res. 2000; 86: 841-5) and that humans with genetic variants giving increased levels of natriuretic peptides have lower blood pressure (Newton-Cheh et al. Nat Genet. 2009; 41: 348-53).

Previous studies have shown that in patients with hypertension there are reduced levels of BNP and ANP and reduced NPR-A activation (Belluardo et al. American Journal of Physiology. Heart and Circulatory Physiology. 2006; 291: H 1529-35; and Macheret et al. J Am Coll Cardiol. 2012; 60: 1558-65). Consequently, recombinant BNP as a replacement therapy has been studied. In a proof-of-concept study, recombinant BNP was shown to give a sustained blood pressure reduction without the need for additional anti-hypertensive drugs in uncontrolled hypertension (Cataliotti et al., Mayo Clinic proceedings. 2012; 87: 413-5).

In addition to the hypotensive (i.e. blood pressure lowering) effects of ANP and BNP, the activation of NPR-A is associated with several beneficial cardiovascular and renal effects, such as inhibition of cardiac hypertrophy, inhibition of the renin-angiotensin-aldosterone system and increased natriuresis and diuresis (see FIG. 1) (Kuhn Physiol Rev. 2016; 96: 751-804). Together these favorable actions can reduce the risk of cardiovascular morbidity and mortality in patients with hypertension and RHT. NPR-A activators could also be a potential treatment of heart failure.

The endogenous natriuretic peptides ANP and BNP have poor bioavailability and a short half-life, resulting in severe challenges to their use as an effective treatment option in hypertension. There has been much effort to stimulate the NPR-A receptor. In recent years, the focus has been on designing natriuretic peptides with improved safety and biological half-life (Meems et al., JACC. Basic to translational science. 2016; 1: 557-567). However, peptides usually have low metabolic stability and short half-life and must be given by injection. This is inconvenient for many patients and reduces patient compliance.

Small peptide-like molecules have also been proposed to stimulate the NPR-A receptor, however these molecules bind to the orthosteric site of the receptor, i.e. the site where the endogenous ligand binds (Iwaki et al. Bioorg Med Chem Lett. 2017; 27: 4904-4907; Iwaki et al. Bioorganic & Medicinal Chemistry. 2017; 25: 1762-1769; and Iwaki et al. Bioorganic & Medicinal Chemistry. 2017; 25: 6680-6694).

The inventors now propose a new mechanism of action for the treatment of hypertension using small molecule drugs as effective NPR-A activators, but which in contrast to previous proposals are allosteric enhancers, i.e. they bind to a different site than the endogenous ligand. Thus, the compounds do not compete with the endogenous ligand for this receptor, but rather enhance the endogenous effects. Although an earlier published abstract proposed allosteric enhancers of NPR-A for the potential treatment of heart failure, this abstract does not reveal the binding site or structures of the molecules (Burnett Journal of Cardiac Failure. 2017; 23: S19).

In contrast to the earlier peptide-like molecules, the small molecule drugs which are proposed herein have a longer biological half-life. As a result they may be given orally to activate NPR-A, increasing patient convenience and compliance. A safety advantage of the allosteric enhancers which are the subject of the present disclosure is a reduced risk of severe side-effects compared to current anti-hypertensives because their effect is achieved by enhancing the actions of endogenous BNP and ANP.

Entresto® (valsartan/sacubitril), also targeting the natriuretic peptide system, has recently been approved for the treatment of congestive heart failure (Ponikowski et al., Eur J Heart Fail. 2016; 18: 891-975). This is a combination therapy that blocks the angiotensin receptor and inhibits a natriuretic peptide-degrading enzyme, called neprilysin. However, in addition to natriuretic peptides, neprilysin is known to degrade other peptides with opposite effects on blood pressure. In contrast to neprilysin inhibitors, the small molecule compounds now proposed by the inventors only affect the direct effects of the natriuretic peptide system and will not have the off-target effects seen when inhibiting neprilysin.

As allosteric enhancers of NPR-A, the compounds herein proposed increase the efficacy and potency of the natriuretic peptides BNP and ANP in their ability to activate NPR-A and thus to generate cGMP. As such, these find use in the treatment of hypertension. Due to their ability to activate NPR-A, the compounds herein described are also considered suitable for the treatment or prevention of other conditions which involve NPR-A including, but not limited to, certain cardiovascular and renal conditions, for example pulmonary hypertension, pre-eclampsia, diabetes, heart failure, cardiovascular disease, cardiac fibrosis and hypertrophy, and diseases associated with fibrosis in other organs and tissues such as the kidneys and lungs.

In one aspect, the invention provides compounds of formula (I), stereoisomers, and pharmaceutically acceptable salts thereof for use in the treatment or prevention of a condition or disorder which is mediated by the natriuretic peptide receptor-A, in particular for use in the treatment or prevention of hypertension, more preferably resistant hypertension:

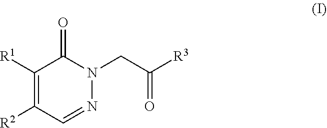

wherein:
R¹ and R² are independently selected from:
H
halogen (e.g. F, Cl or Br),
C₁₋₆ alkyl (e.g. C₁₋₃ alkyl),
C₁₋₆ haloalkyl (e.g. C₁₋₃ haloalkyl), and
CN,
   provided that at least one of R¹ and R² is other than —H;
   R³ is a group of the formula:

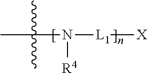

in which n is 0 or 1;
R⁴ is H or C₁₋₃ alkyl (e.g. —CH₃);
L₁ is a linking group selected from:
a bond,
—(CH₂)ₚ— in which p is an integer from 1 to 3, preferably 1 or 2, and
—C₃₋₆ cycloalkylene (e.g. C₃₋₄ cycloalkylene);
   and
   X is selected from:
an aryl group optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and —CN;
a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and optionally substituted phenyl (e.g. unsubstituted phenyl); and
a cycloalkyl group (e.g. a tricyclic cycloalkyl group) optionally substituted by one or more substituents selected from C₁₋₃ alkyl (e.g. —CH₃), C₁₋₃ haloalkyl (e.g. —CF₃), and halogen (e.g. F, C or Br).

In a preferred aspect the invention provides compounds of formula (I), stereoisomers, and pharmaceutically acceptable salts thereof for use in the treatment or prevention of hypertension, in particular for use in the treatment or prevention of resistant hypertension.

In another aspect the invention provides compounds of formula (I), stereoisomers, and pharmaceutically acceptable salts thereof for use as a medicament.

In a further aspect the invention provides a pharmaceutical composition comprising a compound of formula (I), or a stereoisomer, or pharmaceutically acceptable salt thereof, together with one or more pharmaceutically acceptable carriers, excipients or diluents.

In a further aspect the invention provides a pharmaceutical composition comprising a compound of formula (I), or a stereoisomer, or pharmaceutically acceptable salt thereof, together with one or more additional drug substances such as an additional anti-hypertensive agent.

In another aspect the invention relates to novel compounds of formula (II), (IIa), (III) and (IV) as herein described, their stereoisomers, and their pharmaceutically acceptable salts.

In a further aspect the invention relates to a compound of formula (II), (IIa), (III) or (IV) as herein described, or a stereoisomer, or pharmaceutically acceptable salt thereof for use as a medicament.

In a further aspect the invention relates to a compound of formula (II), (IIa), (III) or (IV) as herein described, or a stereoisomer, or pharmaceutically acceptable salt thereof for use in the treatment or prevention of a condition or disorder which is mediated by the natriuretic peptide receptor-A, in particular hypertension, more preferably resistant hypertension.

In a further aspect the invention relates to a process for the preparation of a compound of formula (II), (IIa), (III) or (IV) as herein described, or a stereoisomer, or pharmaceutically acceptable salt thereof.

In a further aspect the present invention provides a pharmaceutical composition comprising a compound of formula (II), (IIa), (III) or (IV) as herein described, or a stereoisomer, or pharmaceutically acceptable salt thereof, together with one or more pharmaceutically acceptable carriers, excipients or diluents.

In a further aspect the invention provides a pharmaceutical composition comprising a compound of formula (II), (IIa), (III) or (IV) as herein described, or a stereoisomer, or pharmaceutically acceptable salt thereof, together with one or more additional drug substances.

Use of any of the compounds herein described, or a stereoisomer, or pharmaceutically acceptable salt thereof, in the manufacture of a medicament for use in the treatment or prevention of a condition or disorder which is mediated by the natriuretic peptide receptor-A, in particular hypertension or, more preferably, resistant hypertension, forms a further aspect of the invention.

A method of treating or preventing a condition or disorder which is mediated by the natriuretic peptide receptor-A, in particular hypertension or, more preferably, resistant hypertension, said method comprising the step of administering to a patient in need thereof (e.g. a human subject) a pharmaceutically effective amount of any compound as herein described, or a stereoisomer, or pharmaceutically acceptable salt thereof, forms a yet further aspect of the invention.

Specific description of the invention

### Definitions

The term “alkyl” as used herein refers to a monovalent saturated, linear or branched, carbon chain. Examples of alkyl groups include, but are not limited to, methyl, ethyl, n-propyl, iso-propyl, n-butyl, iso-butyl, sec-butyl, tert-butyl, n-pentyl, iso-pentyl, neo-pentyl, n-hexyl, etc. An alkyl group preferably contains from 1 to 6 carbon atoms, e.g. 1 to 4 carbon atoms.

The term “alkoxy” refers to an —O-alkyl group, wherein alkyl is as defined herein. Examples of alkoxy groups include, but are not limited to, methoxy, ethoxy, propyloxy, etc.

The term “cycloalkyl” refers to a monovalent, saturated cyclic carbon system. It includes monocyclic, bicyclic and tricyclic rings. Where these contain bicyclic or tricyclic rings, the rings may be linked by a bond or these may be fused. Typically, they will be fused. Monocyclic rings may contain from 3 to 8 carbon atoms, bicyclic and tricyclic rings may contain from 7 to 14 carbon atoms. Examples of monocyclic cycloalkyl groups include, but are not limited to, cyclopropyl, cyclobutyl, cyclopentyl, cyclohexyl, cycloheptyl, etc. Examples of tricyclic rings include adamantyl.

The term “cycloalkylene” refers to a divalent, saturated cyclic carbon system. It includes monocyclic rings containing from 3 to 6 carbon atoms, preferably from 3 to 5 carbon atoms, e.g. 3 or 4 carbon atoms. Examples of monocyclic cycloalkylene groups include, but are not limited to, cyclopropylene and cyclobutylene.

The terms “halogen” and “halogen atom” are used interchangeably herein and preferably refer to F, Cl or Br.

The term “haloalkyl” refers to an alkyl group as defined herein in which at least one of the hydrogen atoms of the alkyl group is replaced by a halogen atom, preferably F, Cl or Br. Examples of such groups include —CH₂F, —CHF₂, —CF₃, —CCl₃, —CHCl₂, —CH₂CF₃, etc.

The term “heterocyclic ring” as used herein refers to a saturated or partially unsaturated carbocyclic system in which at least one ring atom is a heteroatom selected from nitrogen, oxygen and sulfur, the remaining ring atoms being carbon.

The heterocyclic ring structure may be linked to the remainder of the molecule through a carbon atom or through a nitrogen atom.

The terms “aryl” and “aromatic ring” are used interchangeably herein and refer to aromatic ring systems. Such ring systems may be monocyclic or bicyclic and contain at least one aromatic ring. Where such systems contain more than one ring, at least one ring may thus be non-aromatic. Where these contain bicyclic rings, these may be linked by a bond or these may be fused. Any fused bicyclic ring system may contain an aromatic ring fused to a non-aromatic ring (e.g. to a 5- or 6-membered unsaturated carbocyclic ring). Preferably any aryl group will contain from 6-20 carbon atoms, e.g. either 6 or 10 carbon atoms. Examples of “aryl” groups include, but are not limited to, phenyl, 1-napthyl and 2-napthyl. A preferred aryl group is phenyl.

The terms “heteroaryl” and “heteroaromatic ring” are used interchangeably herein and refer to heterocyclic aromatic groups. Such groups may be monocyclic or bicyclic and contain at least one unsaturated heteroaromatic ring system. Where these are monocyclic, these may comprise 5- or 6-membered rings which contain at least one heteroatom selected from nitrogen, oxygen and sulfur and contain sufficient conjugated bonds to form an aromatic system. Where these are bicyclic, these may contain from 9-11 ring atoms. Examples of heteroaryl groups include thiophenyl, thienyl, pyridyl, thiazolyl, furyl, pyrrolyl, triazolyl, imidazolyl, oxadiazolyl, oxazolyl, pyrazolyl, imidazolonyl, oxazolonyl, thiazolonyl, tetrazolyl, thiadiazolyl, benzimidazolyl, benzooxazolyl, benzofuryl, indolyl, isoindolyl, pyridonyl, pyridazinyl, pyrimidinyl, imidazopyridyl, oxazopyridyl, thiazolopyridyl, imidazopyridazinyl, oxazolopyridazinyl, thiazolopyridazinyl and purinyl.

Unless otherwise stated, all substituents are independent of one another.

In the case where a subscript is the integer 0 (i.e. zero), it is intended that the group to which the subscript refers is absent, i.e. there is a direct bond between the groups on either side of that particular group.

Certain compounds herein described may contain one or more stereocenters and may therefore exist in different stereoisomeric forms. The term “stereoisomer” refers to compounds which have identical chemical constitution but which differ in respect of the spatial arrangement of the atoms or groups. Examples of stereoisomers are enantiomers and diastereomers (also called “diastereoisomers”).

The term “enantiomers” refers to two stereoisomers of a compound which are non-superimposable mirror images of one another. The term “diastereomers” or “diastereoisomers” refers to stereoisomers with two or more stereocentres which are not mirror images of one another. The invention is considered to extend to diastereomers and enantiomers, as well as racemic mixtures and enantio-enriched mixtures in which the ratio of enantiomers is other than 1:1.

The compounds herein described may be resolved into their enantiomers and/or diastereomers. For example, where these contain only one stereocenter, these may be provided in the form of a racemate or racemic mixture (a 50:50 mixture of enantiomers) or may be provided as pure enantiomers, i.e. in the R- or S-form. Any of the compounds which occur as racemates may be separated into their enantiomers by methods known in the art, such as column separation on chiral phases or by recrystallization from an optically active solvent. Those compounds with at least two asymmetric carbon atoms may be resolved into their diastereomers on the basis of their physical-chemical differences using methods known per se, e.g. by chromatography and/or fractional crystallization, and where these compounds are obtained in racemic form, they may subsequently be resolved into their enantiomers.

The term “pharmaceutically acceptable salt” as used herein refers to any pharmaceutically acceptable organic or inorganic salt of any of the compounds herein described. A pharmaceutically acceptable salt may include one or more additional molecules such as counter-ions. The counter-ions may be any organic or inorganic group which stabilizes the charge on the parent compound. If the compound of the invention is a base, a suitable pharmaceutically acceptable salt may be prepared by reaction of the free base with an organic or inorganic acid. If the compound of the invention is an acid, a suitable pharmaceutically acceptable salt may be prepared by reaction of the free acid with an organic or inorganic base.

The term “pharmaceutically acceptable” means that the compound or composition is chemically and/or toxicologically compatible with other components of the formulation or with the patient (e.g. human) to be treated.

By “a pharmaceutical composition” is meant a composition in any form suitable to be used for a medical purpose.

As used herein, “treatment” includes any therapeutic application that can benefit a human or non-human animal (e.g. a non-human mammal). Both human and veterinary treatments are within the scope of the present invention, although primarily the invention is aimed at the treatment of humans. Treatment may be in respect of an existing disease or condition or it may be prophylactic.

As used herein, a “pharmaceutically effective amount” relates to an amount that will lead to the desired pharmacological and/or therapeutic effect, i.e. an amount of the agent which is effective to achieve its intended purpose. While individual patient needs may vary, determination of optimal ranges for effective amounts of the active agent is within the capability of one skilled in the art. Generally, the dosage regimen for treating a disease or condition with any of the compounds described herein is selected in accordance with a variety of factors including the nature of the medical condition and its severity.

As used herein, “natriuretic peptide receptor-A” or “NPR-A” refers to a membrane-bound guanylyl cyclase that serves as the receptor for both atrial and brain natriuretic peptides (ANP and BNP, respectively). It may also be known as “guanylyl cyclase A” or “GC-A”. When activated by ANP and BNP, the natriuretic peptide receptor-A causes the production of 3′,5′-cyclic guanosine monophosphate (cGMP) which, via a number of different mechanisms, reduces blood pressure (see FIG. 1).

Any reference herein to “activation of the natriuretic peptide receptor-A” relates to an enhancement of its enzymatic activity to produce cGMP. Reference to an activator (or enhancer) of the natriuretic peptide receptor-A should be construed accordingly. An activator of natriuretic peptide receptor-A is thus a compound that increases its activity and thus increases the production of cGMP.

“Hypertension” as referred to herein, and which may also be referred to as “high blood pressure”, is a long-term medical condition characterized by persistently high blood pressure in the arteries. Long-term complications from high blood pressure include coronary artery disease, stroke, heart failure, atrial fibrillation, peripheral arterial disease, chronic kidney disease, dementia, retinopathy and loss of vision.

According to WHO, hypertension is diagnosed if, when it is measured on two different days in an adult (i.e. a human subject 18 years or older), the systolic blood pressure readings on both days is ≥140 mmHg and/or the diastolic blood pressure readings on both days is ≥90 mmHg. Hypertension is further defined, for example, in “2018 ESC/ESH Guidelines for the management of arterial hypertension” (European Heart Journal, 39 (33): 3021-3104, 1 Sep. 2018), the content of which is incorporated herein by reference.

The invention is based, at least in part, on the finding that certain pyridazinone derivatives are able to target the natriuretic peptide receptor-A causing the production of cGMP. This discovery leads to the use of such compounds to treat or prevent conditions or diseases in subjects, e.g. in humans, which are mediated by the activity of NPR-A. As activators of NPR-A, the compounds herein described are suitable for treating or preventing conditions or diseases which are associated with reduced NPR-A activation, such as hypertension, in particular resistant hypertension.

In one aspect, the invention provides compounds of formula (I), stereoisomers, or pharmaceutically acceptable salts thereof for use in the treatment or prevention of a condition or disorder which is mediated by the natriuretic peptide receptor-A, in particular for use in the treatment of hypertension, more preferably resistant hypertension:

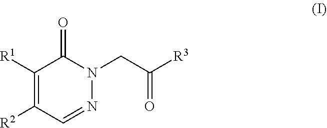

wherein:
R¹ and R² are independently selected from:
H,
halogen (e.g. F, Cl or Br),
C₁₋₆ alkyl (e.g. C₁₋₃ alkyl),
C₁₋₆ haloalkyl (e.g. C₁₋₃ haloalkyl), and
CN,
   provided that at least one of R¹ and R² is other than —H;
   R³ is a group of the formula:

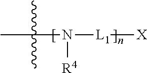

in which n is 0 or 1;
R⁴ is H or C₁₋₃ alkyl (e.g. —CH₃);
L₁ is a linking group selected from:
a bond,
—(CH₂)ₚ— in which p is an integer from 1 to 3, preferably 1 or 2, and
C₃₋₆ cycloalkylene (e.g. C₃₋₄ cycloalkylene);
   and
   X is selected from:
an aryl group optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), and —CN;
a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), and optionally substituted phenyl (e.g. unsubstituted phenyl); and
a cycloalkyl group (e.g. a tricyclic cycloalkyl group) optionally substituted by one or more substituents selected from C₁₋₃ alkyl (e.g. —CH₃), C₁₋₃ haloalkyl (e.g. —CF₃), and halogen (e.g. F, C or Br).

In formula (I) at least one of R¹ and R² in formula (I) is other than hydrogen, i.e. R¹ and R² cannot both be hydrogen.

In one embodiment, R¹ and R² are independently selected from H, and halogen (e.g. F, Cl or Br), preferably from H, Cl and Br. For example, at least one of R¹ and R² may be halogen (e.g. F, Cl or Br), preferably Cl or Br.

In one embodiment, R¹ and R² are both halogen, for example they may both be Cl or Br. In one embodiment, both R¹ and R² are Cl.

In another embodiment, one of R¹ and R² is halogen (e.g. F, Cl or Br) and the other is hydrogen. In one embodiment, R¹ can be halogen and R² can be hydrogen. For example, R¹ can be Cl and R² can be hydrogen.

In formula (I), n is either 0 or 1. Where n is 0, group X is directly linked to the carbonyl group forming a ketone. Where n is 1, group R³ includes a nitrogen atom which is directly linked to the carbonyl group in formula (I) forming an amide.

In one embodiment, the compounds for use in the invention are those of formula (Ia), their stereoisomers, and pharmaceutically acceptable salts:

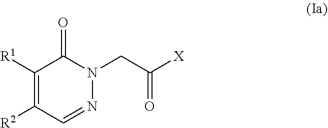

wherein:
R¹ and R² are as herein defined; and
X is selected from:
an aryl group optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and —CN;
a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and optionally substituted phenyl (e.g. unsubstituted phenyl); and
a cycloalkyl group (e.g. a tricyclic cycloalkyl group) optionally substituted by one or more substituents selected from C₁₋₃ alkyl (e.g. —CH₃), C₁₋₃ haloalkyl (e.g. —CF₃), and halogen (e.g. F, Cl or Br).

In another embodiment, the compounds for use in the invention are those of formula (Ib), their stereoisomers, and pharmaceutically acceptable salts:

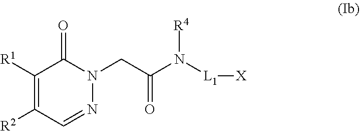

wherein:
R¹, R², R⁴ and L₁ are as herein defined; and
X is selected from:
an aryl group optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), and —CN; and
a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and optionally substituted phenyl (e.g. unsubstituted phenyl).

In an embodiment, the compounds for use in the invention are those of formula (Ic), their stereoisomers, and pharmaceutically acceptable salts:

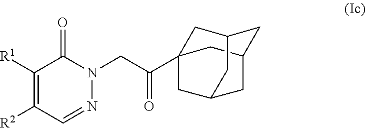

wherein:
R¹ and R² are as herein defined.

In another embodiment, the compounds for use in the invention are those of formula (Id), their stereoisomers, and pharmaceutically acceptable salts:

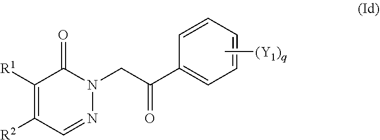

wherein:
R¹ and R² are as herein defined;
each Y₁ is independently selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and —CN; and
q is an integer from 0 to 3, preferably 1 or 2.

In another embodiment, the compounds for use in the invention are those of formula (Ie), their stereoisomers, and pharmaceutically acceptable salts:

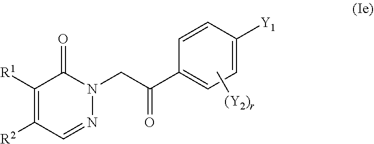

wherein:
R¹ and R² are as herein defined;
Y₁ is as herein defined, preferably C₁₋₃ alkoxy (e.g. —OCH₃), C₁₋₃ haloalkyl (e.g. —CF₃), halogen (e.g. F, C or Br), or —CN;
each Y₂ is independently selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), or —CN, preferably halogen (e.g. F, C or Br); and
r is an integer from 0 to 2, preferably 0 or 1.

In another embodiment, the compounds for use in the invention are those of formula (If), their stereoisomers, and pharmaceutically acceptable salts:

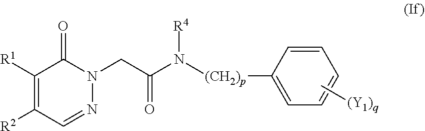

wherein:
R¹, R², R⁴ and p are as herein defined; and
each Y₁ is independently selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and —CN; and
q is an integer from 0 to 3, preferably 1 or 2.

In another embodiment, the compounds for use in the invention are those of formula (Ig), their stereoisomers, and pharmaceutically acceptable salts:

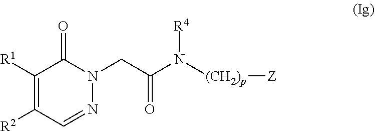

wherein:
R¹, R², R⁴ and p are as herein defined; and
Z is a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), and optionally substituted phenyl (e.g. unsubstituted phenyl).

Examples of compounds for use in accordance with the invention include, but are not limited to, the following, their stereoisomers, and their pharmaceutically acceptable salts:

| Compound | |
|---|---|
| (Example No.) | Structure |
| 1 | 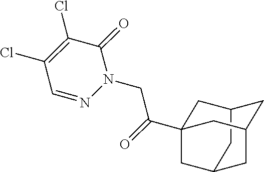 [SMILES: O=C(Cn1ncc(Cl)c(Cl)c1=O)C12CC3CC(CC(C3)C1)C2] |
| 2 | 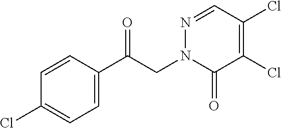 [SMILES: O=C(Cn1ncc(Cl)c(Cl)c1=O)c1ccc(Cl)cc1] |
| 3 (Example 1) | 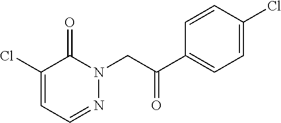 [SMILES: O=C(Cn1nccc(Cl)c1=O)c1ccc(Cl)cc1] |
| 4 (Example 2) | 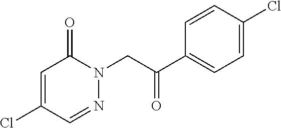 [SMILES: O=C(Cn1ncc(Cl)cc1=O)c1ccc(Cl)cc1] |
| 5 (Example 3) | 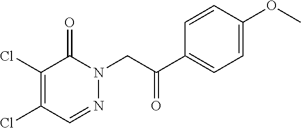 [SMILES: COc1ccc(C(=O)Cn2ncc(Cl)c(Cl)c2=O)cc1] |
| 6 (Example 4) | 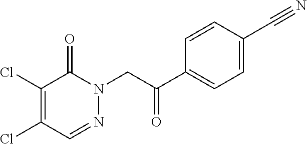 [SMILES: N#Cc1ccc(C(=O)Cn2ncc(Cl)c(Cl)c2=O)cc1] |
| 7 | 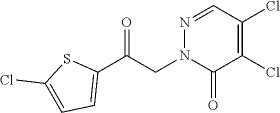 [SMILES: O=C(Cn1ncc(Cl)c(Cl)c1=O)c1ccc(Cl)s1] |
| 8 (Example 5) | 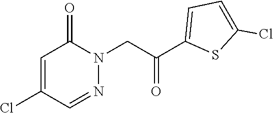 [SMILES: O=C(Cn1ncc(Cl)cc1=O)c1ccc(Cl)s1] |
| 9 | 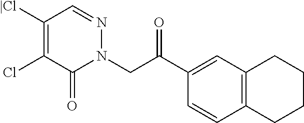 |
| 10 | 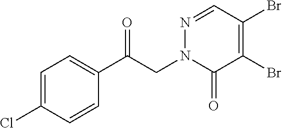 [SMILES: O=C(Cn1ncc(Br)c(Br)c1=O)c1ccc(Cl)cc1] |
| 11 (Example 6) | 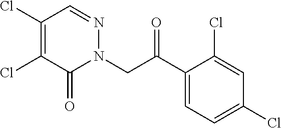 [SMILES: O=C(Cn1ncc(Cl)c(Cl)c1=O)c1ccc(Cl)cc1Cl] |
| 12 (Example 7) | 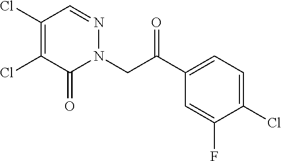 [SMILES: O=C(Cn1ncc(Cl)c(Cl)c1=O)c1ccc(Cl)c(F)c1] |
| 13 (Example 8) | 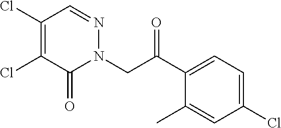 [SMILES: Cc1cc(Cl)ccc1C(=O)Cn1ncc(Cl)c(Cl)c1=O] |
| 14 (Example 9) | 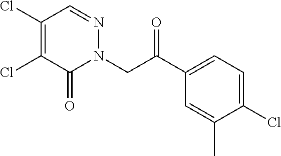 [SMILES: Cc1cc(C(=O)Cn2ncc(Cl)c(Cl)c2=O)ccc1Cl] |
| 15 (Example 10) | 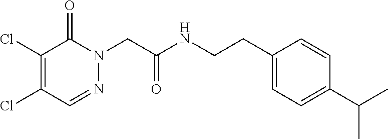 [SMILES: CC(C)c1ccc(CCNC(=O)Cn2ncc(Cl)c(Cl)c2=O)cc1] |
| 16 (Example 11) | 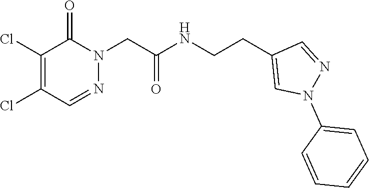 [SMILES: O=C(Cn1ncc(Cl)c(Cl)c1=O)NCCc1cnn(-c2ccccc2)c1] |
| 17 (Example 12) | 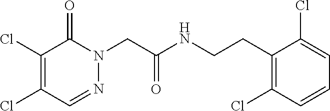 [SMILES: O=C(Cn1ncc(Cl)c(Cl)c1=O)NCCc1c(Cl)cccc1Cl] |
| 18 (Example 13) | 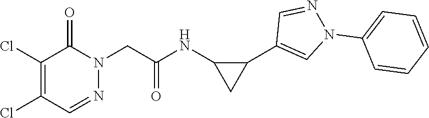 [SMILES: O=C(Cn1ncc(Cl)c(Cl)c1=O)NC1CC1c1cnn(-c2ccccc2)c1] |
| 19 (Example 14) | 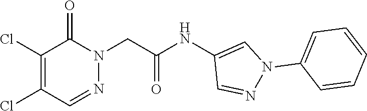 [SMILES: O=C(Cn1ncc(Cl)c(Cl)c1=O)Nc1cnn(-c2ccccc2)c1] |
| 20 (Example 15) | 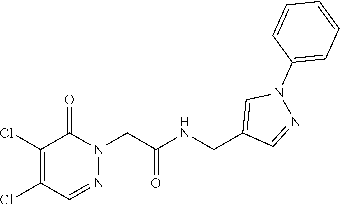 [SMILES: O=C(Cn1ncc(Cl)c(Cl)c1=O)NCc1cnn(-c2ccccc2)c1] |
| 21 | 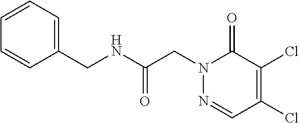 [SMILES: O=C(Cn1ncc(Cl)c(Cl)c1=O)NCc1ccccc1] |
| 22 | 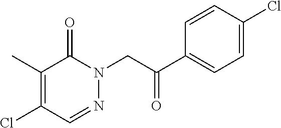 [SMILES: Cc1c(Cl)cnn(CC(=O)c2ccc(Cl)cc2)c1=O] |
| 23 | 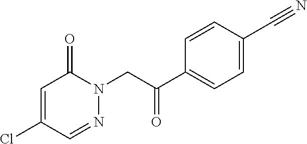 [SMILES: N#Cc1ccc(C(=O)Cn2ncc(Cl)cc2=O)cc1] |
| 24 (Example 16) | 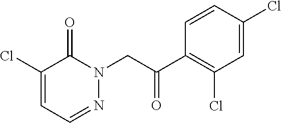 [SMILES: O=C(Cn1nccc(Cl)c1=O)c1ccc(Cl)cc1Cl] |

Compounds preferred for use in the invention are Compound Nos. 1, 2, 11 and 24.

Certain compounds described herein are novel and these form a further aspect of the invention. Thus, in a further aspect, the present invention provides the following compounds of general formula (II), (III) and (IV), their stereoisomers, and pharmaceutically acceptable salts thereof:

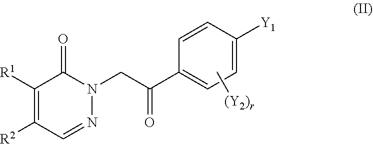

wherein:
R¹ and R² are as herein defined;
Y₁ is as herein defined, preferably C₁₋₃ alkoxy (e.g. —OCH₃), C₁₋₃ haloalkyl, halogen (e.g. F, Cl or Br), or —CN;
each Y₂ is independently selected from C₁₋₃ alkyl (e.g. —CH₃), C₁₋₃ alkoxy (e.g. —OCH₃), C₁₋₃ haloalkyl, halogen (e.g. F, Cl or Br), or —CN, preferably halogen (e.g. F, Cl or Br); and
r is an integer from 0 to 2, preferably 0 or 1;
with the proviso that the compound is other than:
4-chloro-2-[2-(2,4-dichlorophenyl)-2-oxo-ethyl]pyridazin-3-one;
4,5-dichloro-2-[2-(4-chlorophenyl)-2-oxo-ethyl]pyridazin-3-one;
4,5-dichloro-2-[2-(4-methoxyphenyl)-2-oxo-ethyl]pyridazin-3-one; or
4,5-dichloro-2-[2-(4-methylphenyl)-2-oxo-ethyl]pyridazin-3-one.

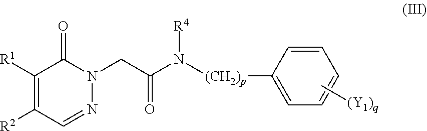

wherein:
R¹, R², R⁴ and p are as herein defined; and
each Y₁ is independently selected from C₁₋₃ alkyl (e.g. —CH₃), C₁₋₃ alkoxy (e.g. —OCH₃), C₁₋₃ haloalkyl, halogen (e.g. F, C or Br), and —CN; and
q is an integer from 0 to 3, preferably 1 or 2;
with the proviso that the compound is other than:
4,5-dichloro-N-[(2,4-dichlorophenyl)methyl]-6-oxo-1(6H)-pyridazineacetamide or
4,5-dichloro-N-[2-(2,4-dichlorophenyl)ethyl]-6-oxo-1(6H)-pyridazineacetamide.

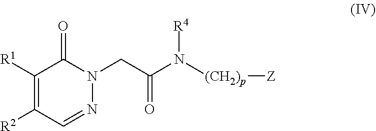

wherein:
R¹, R², R⁴ and p are as herein defined; and
Z is a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and optionally substituted phenyl (e.g. unsubstituted phenyl);
with the proviso that the compound is other than:
4,5-dichloro-N-[[4-(2-methylpropyl)-2-morpholinyl]methyl]-6-oxo-1(6H)-pyridazineacetamide;
4,5-dichloro-N-[2-(1H-indol-3-yl)ethyl]-6-oxo-1(6H)-pyridazineacetamide;
4,5-dibromo-6-oxoN-(4-pyridinylmethyl)-1(6H)-pyridazineacetamide;
4,5-dichloro-N-[(3-methyl-4-pyridinyl)methyl]-6-oxo 1(6H)-pyridazineacetamide;
4,5-dichloro-6-oxo-N-(4-pyridinylmethyl)-1(6H)-pyridazineacetamide;
4,5-dibromo-N-[(3-methyl-4-pyridinyl)methyl]-6-oxo-1(6H)-pyridazineacetamide;
4,5-dichloro-N-[[5-(4-chlorophenyl)-2-furanyl]methyl]-6-oxo-1(6H)-pyridazineacetamide;
4,5-dichloro-6-oxo-N-(2-thienylmethyl)-1(6H)-pyridazineacetamide;
4,5-dibromo-N-[[4-(2-methylpropyl)-2-morpholinyl]methyl]-6-oxo-1(6H)-pyridazineacetamide;
4,5-dichloro-N-(2-furanylmethyl)-6-oxo-1(6H)-pyridazineacetamide;
4,5-dichloro-6-oxo-N-[(tetrahydro-2-furanyl)methyl]-1(6H)-pyridazineacetamide; and
4,5-dichloro-N-[(2,3-dihydro-1,4-benzodioxin-2-yl)methyl]-6-oxo-1(6H)-pyridazineacetamide.

In one embodiment, the compounds according to the invention are those of formula (II), (III) or (IV) in which R² is hydrogen. In one embodiment, the compounds according to the invention are those of formula (II), (III) or (IV) in which R² is hydrogen and R¹ is halogen (e.g. Cl).

In one embodiment, the invention provides compounds of general formula (IIa), their stereoisomers, and pharmaceutically acceptable salts thereof:

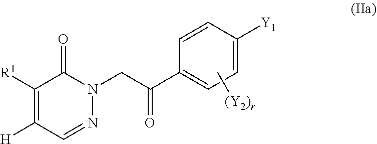

wherein:
R¹ is halogen (e.g. F, Cl or Br), C₁₋₆ alkyl (e.g. C₁₋₃ alkyl), C₁₋₆ haloalkyl (e.g. C₁₋₃ haloalkyl) or —CN, preferably halogen (e.g. F, Cl or Br), e.g. Cl;
Y₁ is as herein defined, preferably C₁₋₃ alkoxy (e.g. —OCH₃), C₁₋₃ haloalkyl, halogen (e.g. F, Cl or Br), or —CN;
each Y₂ is independently selected from C₁₋₃ alkyl (e.g. —CH₃), C₁₋₃ alkoxy (e.g. —OCH₃), C₁₋₃ haloalkyl, halogen (e.g. F, Cl or Br), or —CN, preferably halogen (e.g. F, Cl or Br); and
r is an integer from 0 to 2, preferably 0 or 1.

Novel compounds according to the invention include, but are not limited to, compound Nos. 3, 4, 6, 8, 11, 12, 13, 14, 16, 17, 18, 19, 20 and 24 listed herein, their stereoisomers and pharmaceutically acceptable salts.

In a further aspect the present invention provides a compound of formula (II), (IIa), (III) or (IV), or a stereoisomer, or pharmaceutically acceptable salt thereof, for use as a medicament.

In a further aspect the present invention provides a compound of formula (II), (IIa), (III) or (IV), or a stereoisomer, or pharmaceutically acceptable salt thereof, for use in the treatment or prevention of a condition or disorder which is mediated by the natriuretic peptide receptor-A, in particular hypertension or, more preferably, resistant hypertension.

In a further aspect the present invention provides a pharmaceutical composition comprising a compound of formula (II), (IIa), (III) or (IV), or a stereoisomer, or pharmaceutically acceptable salt thereof, together with one or more pharmaceutically acceptable carriers, excipients or diluents.

In a further aspect the present invention provides a pharmaceutical composition comprising a compound of formula (II), (IIa), (III) or (IV), or a stereoisomer, or pharmaceutically acceptable salt thereof, together with an additional drug substance.

Use of a compound of formula (II), (IIa), (III) or (IV), or a stereoisomer, or pharmaceutically acceptable salt thereof, in the manufacture of a medicament for use in the treatment or prevention of a condition or disorder which is mediated by the natriuretic peptide receptor-A, in particular hypertension or, more preferably, resistant hypertension, forms a further aspect of the invention.

A method of treating or preventing a condition or disorder which is mediated by the natriuretic peptide receptor-A, in particular hypertension, more preferably resistant hypertension, said method comprising the step of administering to a patient in need thereof (e.g. a human subject) a pharmaceutically effective amount of a compound of formula (II), (IIa), (III) or (IV), or a stereoisomer, or pharmaceutically acceptable salt thereof, forms a yet further aspect of the invention.

Any of the compounds herein described may be converted into a salt thereof, particularly into a pharmaceutically acceptable salt thereof with an inorganic or organic acid or base. Procedures for salt formation are conventional in the art.

As will be understood, the compounds described herein may exist in various stereoisomeric forms, including enantiomers, diastereomers, and mixtures thereof.

The invention encompasses all optical isomers of the compounds described herein and mixtures of optical isomers. Hence, compounds that exist as diastereomers, racemates and/or enantiomers are within the scope of the invention.

The compounds of formula (I) are either known in the art, or can be prepared by methods known to those skilled in the art. Some of the compounds are commercially available from sources including Enamine, Sigma-Aldrich and Fluorochem.

Any of the compounds herein described which are not known in the art, including the compounds of formula (II), (IIa), (III) and (IV), may be prepared from readily available starting materials using synthetic methods known in the art such as those described in known textbooks, for example, in Advanced Organic Chemistry (March, Wiley Interscience, 5^{th} Ed. 2001) or Advanced Organic Chemistry (Carey and Sundberg, KA/PP, 4^{th} Ed. 2001).

Schemes 1 and 2 below illustrate general methods suitable for preparing the compounds herein described. Such methods for the preparation of the compounds of formula (II), (la), (III) and (IV) form a further aspect of the invention. The compounds used as starting materials are either known from the literature or may be commercially available. Alternatively, these may readily be obtained by methods known from the literature. As will be understood, other synthetic routes may be used to prepare the compounds using different starting materials, different reagents and/or different reaction conditions. A more detailed description of how to prepare the compounds in accordance with the invention is found in the Examples.

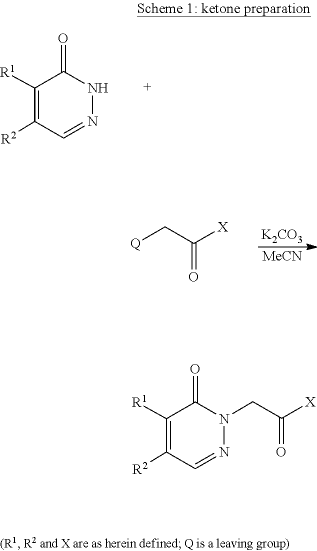

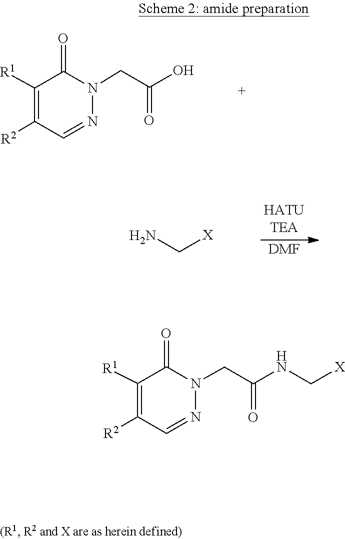

The compounds herein described have valuable pharmacological properties, in particular the ability to activate the natriuretic peptide receptor-A (NPR-A). As such, the compounds are suitable for the treatment or prevention of any condition or disease which is mediated by NPR-A, in particular those conditions or diseases which arise as a result of a reduction in NPR-A activation.

NPR-A plays a central role in maintaining normal blood pressure. The compounds herein described are thus particularly suitable for use as anti-hypertensive agents. In particular, they may be used to treat or prevent primary (essential) hypertension or secondary hypertension. Primary hypertension is defined as high blood pressure which arises due to lifestyle and genetic factors and is the most common type. Lifestyle factors that increase the risk of primary hypertension include excess salt in the diet, excess bodyweight, smoking and alcohol consumption. Secondary hypertension arises due to an identifiable cause, such as chronic kidney disease, narrowing of the kidney arteries, or an endocrine disorder such as Cushing's syndrome, hyperthyroidism, or hypothyroidism. Other causes of secondary hypertension include obesity, pregnancy, excessive alcohol consumption, certain prescription medicines and stimulants such as cocaine and methamphetamine. Pre-eclampsia is also associated with hypertension.

In a particular embodiment, the compounds herein described find use in lowering blood pressure in hypertensive patients that are not responsive to current anti-hypertensive treatments, i.e. in the treatment of so-called “resistant hypertension”. As used herein, the term “resistant hypertension” refers to high blood pressure that remains above a target level (e.g. above 140/90 mm Hg) in a subject (e.g. a human patient) despite the subject taking simultaneously three or more anti-hypertensive drugs having different mechanisms of action. Subjects (e.g. patients) which may benefit from treatment in accordance with the invention thus include those diagnosed as hypertensive and who have previously been prescribed (i.e. undertaken) a treatment involving simultaneous administration of at least three different anti-hypertensive drugs, but who have not responded.

Hypertension can cause serious damage to the heart. Excessive pressure can harden arteries, decreasing the flow of blood and oxygen to the heart. This elevated pressure and reduced blood flow can cause other complications, such as angina, coronary artery disease, heart attack, myocardial infarction, heart failure, and an irregular heart beat which can lead to sudden death. Hypertension can also burst or block arteries that supply blood and oxygen to the brain, causing a stroke. It can also cause kidney damage, which may ultimately lead to kidney failure. Other long-term effects of high blood pressure may include dementia, retinopathy and loss of vision. The compounds herein described may thus also be used to treat, prevent, or reduce the risk of a subject (e.g. a patient) developing any of these listed complications. In one embodiment, the compounds herein described may therefore be used in the treatment or prevention of any of the following conditions: angina pectoris, coronary artery disease, heart attack, myocardial infarction, heart failure, irregular heartbeat, stroke, kidney damage, kidney failure, dementia, retinopathy, and loss of vision.

In one embodiment, the compounds herein described may be used to treat or prevent pulmonary hypertension, pre-eclampsia, diabetes, heart failure or cardiovascular disease. In another embodiment, the compounds may be used to treat or prevent cardiac fibrosis or hypertrophy, or any disease or condition involving fibrosis in other organs or tissues including, but not limited to, the kidneys or the lungs. The treatment of hypertension represents a preferred embodiment of the invention, in particular the treatment of resistant hypertension.

Viewed from a further aspect the invention provides a compound, stereoisomer or pharmaceutically acceptable salt as herein described for use in therapy. Unless otherwise specified, the term “therapy” as used herein is intended to include both treatment and prevention.

In a further aspect the invention provides a compound, stereoisomer or pharmaceutically acceptable salt as herein described for use in the treatment or prevention of any of the conditions herein described, for example in the treatment or prevention of hypertension, more preferably resistant hypertension.

In another aspect the invention provides the use of a compound, stereoisomer or pharmaceutically acceptable salt as herein described in the manufacture of a medicament for use in a method of treatment or prevention of any of the conditions herein described, for example in the treatment or prevention of hypertension, more preferably resistant hypertension.

Also provided is a method of treatment of a human or non-human animal body to combat or prevent any of the conditions herein described, for example to combat or prevent hypertension, more particularly resistant hypertension, said method comprising the step of administering to said body an effective amount of a compound, stereoisomer or pharmaceutically acceptable salt as herein described.

For use in a therapeutic or prophylactic treatment, the compounds herein described will typically be formulated as a pharmaceutical formulation. In a further aspect, the invention thus provides a pharmaceutical composition comprising a compound, stereoisomer or pharmaceutically acceptable salt as herein described, together with one or more pharmaceutically acceptable carriers, excipients or diluents.

Acceptable carriers, excipients and diluents for therapeutic use are well known in the art and can be selected with regard to the intended route of administration and standard pharmaceutical practice. Examples include binders, lubricants, suspending agents, coating agents, solubilizing agents, preserving agents, wetting agents, emulsifiers, surfactants, sweeteners, colorants, flavoring agents, antioxidants, odorants, buffers, stabilizing agents and/or salts.

The compounds herein described may be formulated with one or more conventional carriers and/or excipients according to techniques well known in the art. Typically, the compositions will be adapted for oral or parenteral administration, for example by intradermal, subcutaneous, intramuscular, intraperitoneal or intravenous injection. For parenteral administration, compositions that are adapted for subcutaneous, intramuscular or intravenous administration are generally preferred.

In one embodiment, the compositions herein described are adapted for subcutaneous injection. A typical composition for subcutaneous injection is a sterile aqueous suspension or solution of a compound of formula (I), or a stereoisomer or pharmaceutically acceptable salt thereof.

In one embodiment, the compositions herein described are adapted for intramuscular injection. A typical composition for intramuscular injection is a sterile sustained release formulation in which a compound of formula (I), or a stereoisomer or pharmaceutically acceptable salt thereof, is formulated in an oily suspension.

In one embodiment, the compositions herein described are adapted for intravenous injection. A typical composition for intravenous injection is a sterile aqueous solution of a compound of formula (I), or a stereoisomer or pharmaceutically acceptable salt thereof.

Compositions for injection are preferably isotonic solutions (osmolality around 300 mOsm/kg). Slightly hypertonic solutions (osmolality less than 600 mOsm/kg) are also acceptable. Any injection solution may additionally comprise one or more physiologically acceptable salts or other pharmaceutically acceptable additives, such as for example sugar compounds to provide an acceptable osmolality.

Several of the compounds defined by formula (I) herein have limited aqueous solubility. If any compound of formula (I) is not sufficiently water soluble per se to provide an aqueous injection solution, it is preferably formulated as a pharmaceutically acceptable salt if possible. Other compositions suitable for injection according to the invention are compositions comprising solubilizers. Typical solubilizers are water-soluble co-solvents like for example ethanol, glycerol and polyethylene glycols. Other solubilizers include surfactants like for example polysorbate 80 (Tween 80). A preferred group of solubilizers are the cyclodextrins and their pharmaceutically acceptable salts; preferably 2-hydroxypropyl-beta-cyclodextrin, 4-sulfobutyl-beta-cyclodextrin or sulfobutylether-beta-cyclodextrin, and their pharmaceutically acceptable salts (e.g. their sodium salts). Cyclodextrin complexes of compounds defined by formula (I) form a further aspect of the invention. These can be prepared in situ or in a separate process using a mortar and pestle with minor amounts of water followed by drying of the complex prior to preparation of the composition. Pharmaceutical additives such as solubilizers and methods for preparation of pharmaceutical compositions of compounds with limited aqueous solubility are well described in standard teaching books in pharmaceutical sciences; see for example Remington: The Science and Practice of Pharmacy, 23^{rd} edition, 2020, Academic Press.

In another aspect, the invention provides an aqueous pharmaceutical formulation adapted for injection, said formulation comprising a compound of formula (I), a stereoisomer or a pharmaceutically acceptable salt thereof, together with at least one pharmaceutically acceptable solubilizing agent, for example a cyclodextrin or a pharmaceutically acceptable salt thereof. In one embodiment, the compound of formula (I) present in the formulation is a compound having the formula (IIa) as herein described.

Sterile compositions of any of the compounds herein described can typically be prepared by autoclaving or sterile filtration. The final injection composition may be provided in the form of a pre-filled syringe or as injection vials.

In a preferred embodiment, the compounds may be adapted for oral administration. For example, these may be formulated in conventional oral administration forms, e.g. tablets, coated tablets, capsules, powders, granulates, solutions, dispersions, suspensions, syrups, emulsions, etc. using conventional excipients, e.g. solvents, diluents, binders, sweeteners, aromas, pH modifiers, viscosity modifiers, antioxidants, etc. Suitable excipients may include, for example, corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose or fatty substances such as saturated fats or suitable mixtures thereof, etc.

Preferred dosage forms for oral administration of the compounds are tablets and capsules. Some of the compounds defined by formula (I) may be formulated with additives to improve oral bioavailability. Such additives are well described in standard teaching books in pharmaceutical sciences; see for example Remington: The Science and Practice of Pharmacy, 23rd edition, 2020, Academic Press. Typical such additives include various cyclodextrins (for example, beta-cyclodextrin), surfactants and various hydrophobic excipients. One group of preferred excipients are those capable of forming an emulsion in the gastrointestinal system (i.e. “self-emulsifying” formulations).

The dosage required to achieve the desired activity of the compounds herein described will depend on various factors, such as the compound selected, its mode and frequency of administration, whether the treatment is therapeutic or prophylactic, and the nature and severity of the disease or condition, etc. Typically, a physician will determine the actual dosage which will be most suitable for an individual subject. The specific dose level and frequency of dosage for any particular patient may be varied and will depend upon factors such as the activity of the specific compound employed, the metabolic stability and length of action of that compound, the age of the patient, the mode and time of administration, and the severity of the particular condition. The compound and/or the pharmaceutical composition may be administered in accordance with a regimen from 1 to 6 times per day, such as once or twice per day.

Suitable daily dosages of the compounds herein described are expected to be in the range from 0.1 mg to 1 g of the compound; 1 mg to 500 mg of the compound; 1 mg to 300 mg of the compound; 5 mg to 100 mg of the compound, or 10 mg to 100 mg of the compound. By a “daily dosage” is meant the dosage per 24 hours.

The compounds described herein may be used alone in the treatment of any of the conditions herein described. Alternatively, any of the methods of treatment herein described may advantageously be combined with administration of one or more additional active agents which are effective in treating the disorder or disease to be treated, i.e. as an add-on therapy to current regimes.

In one embodiment, the compounds herein described may be used in combination with one or more conventional anti-hypertensive drugs. Such treatment methods may involve simultaneous, separate or sequential administration of a compound as herein described, or a pharmaceutical composition containing the compound, and the additional anti-hypertensive agent or agents. Where the actives are to be administered simultaneously, these may be provided in the form of a combined preparation. Thus, any of the pharmaceutical compositions herein described may additionally contain one or more of such active agents, for example one or more anti-hypertensive agents.

A preferred combination for use according to the invention is a combination of a compound of formula (I), (II), (IIa), (III) or (IV), or a stereoisomer or pharmaceutically acceptable salt thereof as herein described, together with at least one drug substance having regulatory approval (for example in the United States or the EU) for the treatment of a cardiovascular disease, for example which is approved for the treatment of hypertension or, more preferably, resistant hypertension. Preferred for use in the invention are combinations of drugs having different mechanisms of action. Drugs having different mechanisms of action are those that bind or interfere with different biological targets, or that bind or interfere with different sites on the same biological target. The use of combinations of drugs having different mechanisms of action may be advantageous in that it permits the use of lower doses of the drugs, or improves safety of the treatment with fewer side effects and/or improved efficacy.

Combined preparations for use according to the invention include those adapted for oral administration. In one embodiment, the invention thus provides a combined preparation adapted for oral administration comprising a compound of formula (I), (II), (IIa), (III) or (IV), or a stereoisomer or pharmaceutically acceptable salt thereof as herein described, together with one or more additional drug substances as herein described. In one embodiment, the combined preparation is provided in unit dose form, for example in the form of a tablet or capsule. Suitable unit dosages may have a weight of up to 1,000 mg, for example up to 800 mg, or up to 600 mg.

Examples of anti-hypertensive drugs which may be co-administered with the compounds herein described include inhibitors of the renin-angiotensin-aldosterone system such as angiotensin-converting-enzyme inhibitors (ACE inhibitors), for example benazepril, zofenopril, perindopril, trandolapril, captopril, enalapril, lisinopril, and ramipril; angiotensin II receptor antagonists, for example losartan, telmisartan, irbesartan, candesartan, olmesartan, valsartan and saprisartan; thiazide diuretics, for example hydrochlorothiazide and bendroflumethiazide; thiazide-like diuretics, for example chlorthalidone; calcium channel blockers such as dihydropyrines, for example amlodipine, felodipine, isradipine, nifedipine, nimodipine, lerkanidipine, verapamil or dilthiazem; beta-blockers, for example metoprolol, propranolol, timolol, atenolol, bisoprolol, esmolol, nebivolol and carvedilol; aldosterone antagonists such as spironolactone and eplerenone; and alpha-blockers such as prazosine and tamsulozine. In one embodiment, the anti-hypertensive drug may be a pi-selective beta-blocker such as acebutolol, atenolol, betaxolol, bisoprolol, celiprolol, metoprolol, nebivolol or esmolol. In another embodiment, the anti-hypertensive drug may be a combined alpha- and beta-blocker such as labetalol or carvedilol. Other anti-hypertensive drugs which may be co-administered include sacubitril and sacubritril-valsartan (LCZ596).

In another embodiment, the invention also provides a package comprising: (i) a compound, stereoisomer or pharmaceutically acceptable salt as herein described or a pharmaceutical composition comprising said compound, stereoisomer or pharmaceutically acceptable salt; and (ii) printed instructions and/or a label relating to the use of (i) in the treatment of any of the conditions or disorders as herein described, for example in the treatment or prevention of hypertension, more particularly resistant hypertension, in a subject (e.g. a patient).

The invention further provides a method of combination therapy for the treatment of hypertension, e.g. resistant hypertension, in a subject in need of such treatment, said method comprising the step of administering to said subject a therapeutically effective amount of a compound, stereoisomer or pharmaceutically acceptable salt as herein described, and simultaneously or separately (e.g. sequentially) one or more anti-hypertensive drugs.

The invention also provides a pharmaceutical composition comprising a compound, stereoisomer or pharmaceutically acceptable salt as herein described and one or more anti-hypertensive drugs, e.g. an inhibitor of the renin-angiotensin-aldosterone system, a thiazide diuretic, a calcium channel blocker, or a beta-blocker, optionally in combination with at least one pharmaceutically acceptable carrier or excipient.

The pharmacological properties of the compounds of the invention can be analyzed using standard assays for functional activity. Detailed protocols for testing of the compounds of the invention are provided in the Examples.

### EXAMPLES

The invention will now be described in more detail in the following non-limiting Examples and with reference to the accompanying figure in which:

FIG. 1 schematically illustrates the activation of NPR-A by the action of ANP and BNP causing production of cGMP which, through several mechanisms, reduces blood pressure.

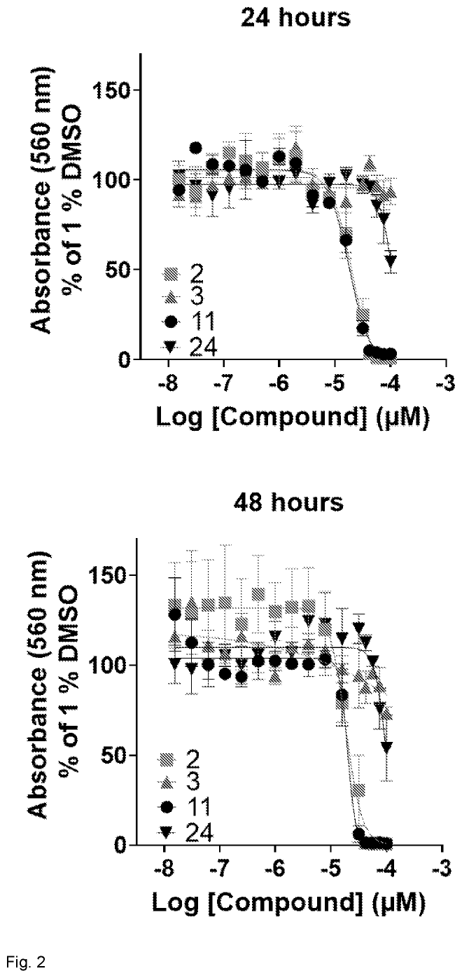

FIG. 2 shows the results from MTT assays performed in HeLa cells. Cells were incubated for 24 and 48 h with increasing concentrations of the indicated compound. The data represent formazan formation (absorbance) as a percentage of the negative control (1% DMSO).

### PREPARATION OF INTERMEDIATES

### Preparation of methyl 2-(4,5-dichloro-6-oxopyridazin-1(6H)-yl)acetate

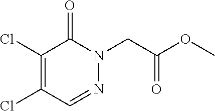

This compound was prepared according to the general procedure A as a white solid (1.60 gram, 85% yield).

LC/MS (ESI) m/z for C₇H₆Cl₂N₂O₃ 236/238 (calcd) 237/239 ([M+H]⁺ found).

¹H NMR (400 MHz, CDCl₃) δ 7.81 (s, 1H), 4.91 (s, 2H), 3.80 (s, 3H).

### Preparation of tert-butyl 2-(4,5-dichloro-6-oxopyridazin-1(6H)-yl)acetate

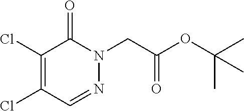 [SMILES: CC(C)(C)OC(=O)Cn1ncc(Cl)c(Cl)c1=O]

This compound was prepared according to the general procedure A as a white solid (1.54 gram, 92% yield).

LC/MS (ESI) m/z for C₁₀H₁₂Cl₂N₂O₃ 278/280 (calcd) 223/225 ([M-t-Bu+H]⁺ found).

¹H NMR (400 MHz, CDCl₃) δ 7.80 (s, 1H), 4.80 (s, 2H), 1.49 (s, 9H).

### Preparation of 2-(4,5-dichloro-6-oxopyridazin-1(6H)-yl)acetic acid

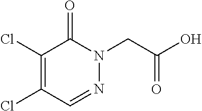 [SMILES: O=C(O)Cn1ncc(Cl)c(Cl)c1=O]

In a 100 ml flask tert-butyl 2-(4,5-dichloro-6-oxopyridazin-1(6H)-yl)acetate (1.54 g, 5.50 mmol, see Example 7) was treated with an excess of hydrogen chloride in dioxane at 70° C. Once the conversion was complete, the mixture was evaporated to dryness, the residue was triturated from heptane, the solids were filtered off on a glass filter providing 1.19 gram of an off-white solid (96% yield).

LC/MS (ESI) m/z for C₆H₄Cl₂N₂O₃ 222/224 (calcd) 223/225 ([M+H]⁺ found).

¹H NMR (400 MHz, CDCl₃) δ 9.38 (very br s, 1H), 7.83 (s, 1H), 4.96 (s, 2H).

### GENERAL PROCEDURES

In the following general procedures groups R¹, R² and X are as herein defined; and Q is a leaving group.

### General Procedure A: Pyridazinone Alkylation

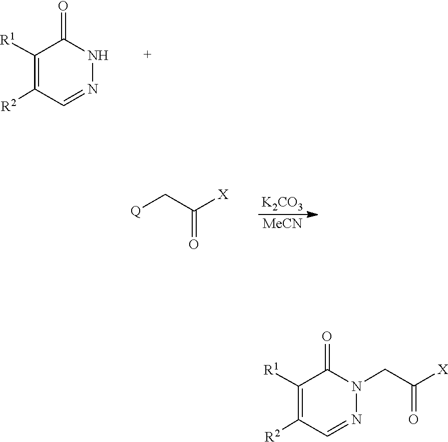

An appropriate pyridazin-3(2H)-one (1.0 equiv.), haloacetophenone or haloacetate (1.2 equiv.) and potassium carbonate (1.5 equiv.) are suspended in acetonitrile (0.05 to 0.20 M). The mixture is heated at 70° C. for 1-3 hours. After absorbing the mixture on isolute and drying, the residue is subjected to flash chromatography, usually eluting with a gradient of ethyl acetate (5% to 50%) in heptane. The fraction of the product is collected and evaporated to dryness resulting in the desired compound.

### General Procedure B: Amide Preparation

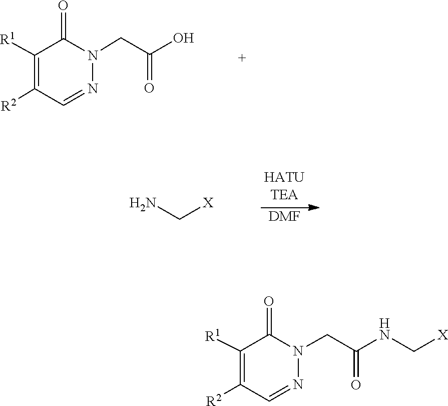

To a solution of 2-(4,5-dichloro-6-oxopyridazin-1(6H)-yl)acetic acid (1.0 equiv.) and triethylamine (1.2 equiv.) in dry DMF (0.10 M) is added HATU (1.1 equiv.) under an inert atmosphere. The solution is stirred for 1 h before the appropriate amine (1.1 equiv.) is added. The stirring is continued for 1 to 4 hours and then directly purified by preparative chromatography, to afford the target amide as a white or off-white solid.

### Example 1—Preparation of 4-chloro-2-(2-(4-chlorophenyl)-2-oxoethyl)pyridazin-3(2H)-one

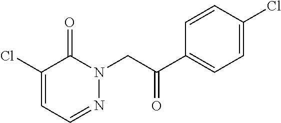 [SMILES: O=C(Cn1nccc(Cl)c1=O)c1ccc(Cl)cc1]

The title compound was prepared according to the general procedure A as a white solid (27 mg, 95% yield).

LC/MS (ESI) m/z for C₁₂H₈Cl₂N₂O₂ 282/284 (calcd) 283/285 ([M+H]⁺ found).

¹H NMR (400 MHz, CDCl₃) δ 7.98-7.90 (m, 2H), 7.75 (d, J=4.3 Hz, 1H), 7.54-7.46 (m, 2H), 7.43 (d, J=4.4 Hz, 1H), 5.59 (s, 2H).

### Example 2—Preparation of 5-chloro-2-(2-(4-chlorophenyl)-2-oxoethyl)pyridazin-3(2H)-one

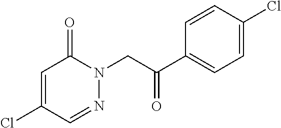 [SMILES: O=C(Cn1ncc(Cl)cc1=O)c1ccc(Cl)cc1]

The title compound was prepared according to the general procedure A as a white solid (27 mg, 95% yield).

LC/MS (ESI) m/z for C₁₂H₈Cl₂N₂O₂ 282/284 (calcd) 283/285 ([M+H]⁺ found).

¹H NMR (400 MHz, CDCl₃) δ 7.96-7.89 (m, 2H), 7.79 (d, J=2.3 Hz, 1H), 7.54-7.46 (m, 2H), 7.03 (d, J=2.4 Hz, 1H), 5.52 (s, 2H).

### Example 3—Preparation of 4,5-dichloro-2-(2-(4-methoxyphenyl)-2-oxoethyl) pyridazin-3(2H)-one

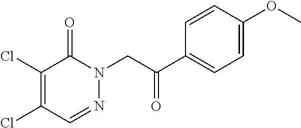 [SMILES: COc1ccc(C(=O)Cn2ncc(Cl)c(Cl)c2=O)cc1]

The title compound was prepared according to the general procedure A as a white solid (30 mg, 93% yield).

LC/MS (ESI) m/z for C₁₃H₁₀Cl₂N₂O₃ 312/314 (calcd) 313/315 ([M+H]⁺ found).

¹H NMR (400 MHz, CDCl₃) δ 8.01-7.92 (m, 2H), 7.84 (s, 1H), 7.02-6.94 (m, 2H), 5.57 (s, 2H), 3.90 (s, 3H).

### Example 4—Preparation of 4-(2-(4,5-dichloro-6-oxopyridazin-1(6H)-yl)acetyl) benzonitrile

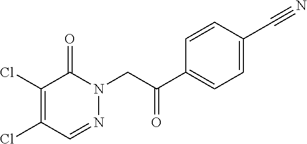 [SMILES: N#Cc1ccc(C(=O)Cn2ncc(Cl)c(Cl)c2=O)cc1]

The title compound was prepared according to the general procedure A as a white solid (28 mg, 89% yield).

LC/MS (ESI) m/z for C₁₃H₇Cl₂N₃O₂ 307/309 (calcd) 308/310 ([M+H]⁺ found).

¹H NMR (400 MHz, CDCl₃) δ 8.12-8.05 (m, 2H), 7.86 (s, 1H), 7.85-7.80 (m, 2H), 5.58 (s, 2H).

### Example 5—Preparation of 5-chloro-2-(2-(5-chlorothiophen-2-yl)-2-oxoethyl) pyridazin-3(2H)-one

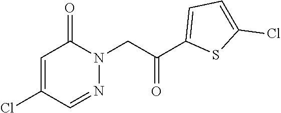 [SMILES: O=C(Cn1ncc(Cl)cc1=O)c1ccc(Cl)s1]

The title compound was prepared according to the general procedure A as a pale yellow solid (19 mg, 93% yield).

LC/MS (ESI) m/z for C₁₀H₆Cl₂N₂O₂S 288/290 (calcd) 289/291 ([M+H]⁺ found).

¹H NMR (400 MHz, CDCl₃) δ 7.78 (d, J=2.4 Hz, 1H), 7.62 (d, J=4.1 Hz, 1H), 7.05-7.00 (m, 2H), 5.40 (s, 2H).

### Example 6—Preparation of 4,5-dichloro-2-[2-(2,4-dichlorophenyl)-2-oxo-ethyl]pyridazin-3-one

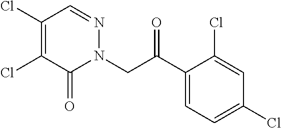 [SMILES: O=C(Cn1ncc(Cl)c(Cl)c1=O)c1ccc(Cl)cc1Cl]

A solution of 4,5-dichloro-2H-pyridazin-3-one (165 mg, 1 mmol) and 1,8-diazabicyclo [5.4.0] undec-7-ene (0.17 mL, 1 mmol,) in N,N-dimethylformamide (5 mL) was cooled to 0° C. A solution of 2,2′,4′-trichloroacetophenone (223 mg, 1 mmol) in N,N-dimethylformamide (3 mL) was added to the reaction mixture dropwise at 0° C. The reaction mixture was allowed to reach room temperature and stirred overnight. After the reaction was completed (controlled by TLC), the solvent was evaporated under reduced pressure. Water (15 mL) was added and the mixture was extracted by ethyl acetate (20 mL). The solution was dried over sodium sulfate and the product was purified using column chromatography (EtOAc/Hexane: 1/3) and the title compound was obtained as a white solid (50 mg after purification, 14%).

¹H NMR (400 MHz, DMSO-d6) δ 5.70 (s, 2H), 7.71-7.73 (d, 1H, J=9.6 Hz), 7.9 (s, 1H), 8.03-8.06 (d, 1H, J=7.6), 8.40 (s, 1H).¹3C NMR (400 MHz, DMSO-d6) δ 61.66, 127.83, 130.70, 131.64, 132.84, 134.22, 134.44, 136.39, 137.25, 139.17, 156.88, 192.07

### Example 7—Preparation of 4,5-dichloro-2-[2-(4-chloro-3-fluoro-phenyl)-2-oxo-ethyl]pyridazin-3-one

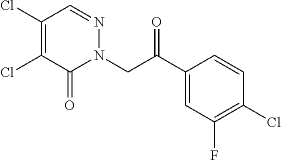 [SMILES: O=C(Cn1ncc(Cl)c(Cl)c1=O)c1ccc(Cl)c(F)c1]

A solution of 4,5-dichloro-2H-pyridazin-3-one (165 mg, 1 mmol) and 1,8-diazabicyclo [5.4.0] undec-7-ene (0.17 mL, 1 mmol,) in N,N-dimethylformamide (5 mL) was cooled to 0° C. A solution of 2-chloro-1-(4-chloro-3-fluorophenyl)ethanone (207 mg, 1 mmol) in N,N-dimethylformamide (3 mL) was added to the reaction mixture dropwise at 0° C. The reaction mixture was allowed to reach room temperature and stirred overnight. After the reaction was completed (controlled by TLC), the solvent was evaporated under reduced pressure. Water (15 mL) was added and the mixture was extracted by ethyl acetate (20 mL). The solution was dried over sodium sulfate and the product was purified using column chromatography (EtOAc/Hexane: 1/3) and the title compound was obtained as a white solid (45 mg after purification, 13%).

¹H NMR (400 MHz, DMSO-d6) δ 5.80 (s, 2H), 7.84-7.87 (t, 1H, J=7.6 Hz), 7.91-9.94 (dd, 1H, J=2.2 Hz), 8.08-8.11 (dd, 1H, J=1.3 Hz), 8.32 (s, 1H)

### Example 8—Preparation of 4,5-dichloro-2-[2-(4-chloro-2-methyl-phenyl)-2-oxo-ethyl]pyridazin-3-one

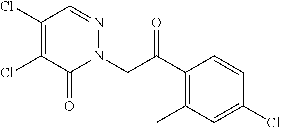 [SMILES: Cc1cc(Cl)ccc1C(=O)Cn1ncc(Cl)c(Cl)c1=O]

A solution of 4,5-dichloro-2H-pyridazin-3-one (165 mg, 1 mmol) and 1,8-diazabicyclo [5.4.0] undec-7-ene (0.17 mL, 1 mmol,) in N,N-dimethylformamide (5 mL) was cooled to 0° C. A solution of 2-chloro-1-(4-chloro-2-methylphenyl)ethanone (203 mg, 1 mmol) in N,N-dimethylformamide (3 mL) was added to the reaction mixture dropwise at 0° C. The reaction mixture was allowed to reach room temperature and stirred overnight. After the reaction was completed (controlled by TLC), the solvent was evaporated under reduced pressure. Water (15 mL) was added and the mixture was extracted by ethyl acetate (20 mL). The solution was dried over sodium sulfate and the product was purified using column chromatography (EtOAc/Hexane: 1/3) and the title compound was obtained as a white solid (16 mg after purification, 5%).

¹H NMR (400 MHz, DMSO-d6) δ 2.43 (s, 3H), 5.63 (s, 2H), 7.48-7.49 (d, 1H, J=7.8 Hz), 8.00-8.03 (d, 1H, J=8.4 Hz), 8.32 (s, 1H)

### Example 9—Preparation of 4,5-dichloro-2-[2-(4-chloro-3-methyl-phenyl)-2-oxo-ethyl]pyridazin-3-one

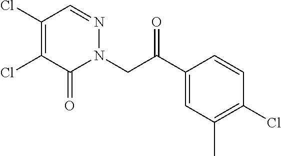 [SMILES: Cc1cc(C(=O)Cn2ncc(Cl)c(Cl)c2=O)ccc1Cl]

A solution of 4,5-dichloro-2H-pyridazin-3-one (165 mg, 1 mmol) and 1,8-diazabicyclo [5.4.0] undec-7-ene (0.17 mL, 1 mmol,) in N,N-dimethylformamide (5 mL) was cooled to 0° C. A solution of 2-chloro-1-(4-chloro-3-methylphenyl)ethanone (203 mg, 1 mmol) in N,N-dimethylformamide (3 mL) was added to the reaction mixture dropwise at 0° C. The reaction mixture was allowed to reach room temperature and stirred overnight. After the reaction was completed (controlled by TLC), the solvent was evaporated under reduced pressure. Water (15 mL) was added and the mixture was extracted by ethyl acetate (20 mL). The solution was dried over sodium sulfate and the product was purified using column chromatography (EtOAc/Hexane: 1/3) and the title compound was obtained as a white solid (47 mg after purification, 14%).

¹H NMR (400 MHz, DMSO-d6) δ 2.43 (s, 3H), 5.76 (s, 2H), 7.64-7.66 (d, 1H, J=7.7 Hz), 7.88-7.91 (dd, 1H, J=8.8, 2.2 Hz), 8.07-8.07 (d, 1H, J=1.7), 8.32 (s, 1H)

### Example 10—Preparation of 2-(4,5-dichloro-6-oxopyridazin-1(6H)-yl)-N-(4-isopropylphenethyl)acetamide

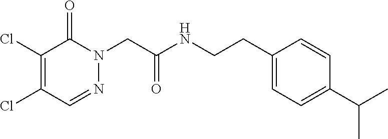 [SMILES: CC(C)c1ccc(CCNC(=O)Cn2ncc(Cl)c(Cl)c2=O)cc1]

In a 10 ml tube 2-(4-isopropylphenyl)ethan-1-amine (0.017 g, 0.105 mmol) was dissolved in dry toluene (1.0 ml) under a nitrogen atmosphere. The solution was treated with 2 M solution in toluene of trimethylaluminium (0.053 ml, 0.105 mmol) observing some fuming. After stirring for 15 minutes, methyl 2-(4,5-dichloro-6-oxopyridazin-1(6H)-yl)acetate (0.024 g, 0.10 mmol) was added and the pale orange mixture was heated at 70° C. overnight. After cooling down and quenching with a drop of 1 N aqueous HCl, the resulting suspension was diluted with water and extracted three times with DCM. The extracts were combined and dried over sodium sulfate, filtered and evaporated leaving a pale orange solid of the crude. Once absorbed on isolute, it was purified by flash chromatography eluting with a gradient of ethyl acetate (10% to 100%) in heptane. The product fraction provided 14 mg (37% yield) of a white solid.

LC/MS (ESI) m/z for C₁₇H₁₉Cl₂N₃O₂ 367/369 (calcd) 368/370 ([M+H]⁺ found).

¹H NMR (400 MHz, CDCl₃) δ 7.81 (s, 1H), 7.18-7.03 (symmetrical m, 4H), 5.92 (br s, 1H), 4.77 (s, 2H), 3.53 (q, J=6.6 Hz, 2H), 2.88 (hept, J=7.1 Hz, 1H), 2.78 (t, J=6.8 Hz, 2H), 1.24 (d, J=6.9 Hz, 6H).

### Example 11—Preparation of 2-(4,5-dichloro-6-oxopyridazin-1(6H)-yl)-N-(2-(1-phenyl-1H-pyrazol-4-yl)ethyl)acetamide

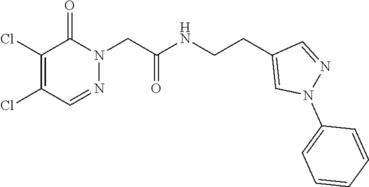 [SMILES: O=C(Cn1ncc(Cl)c(Cl)c1=O)NCCc1cnn(-c2ccccc2)c1]

The title compound was prepared according to the general procedure B as a white solid.

LC/MS (ESI) m/z for C₁₇H₁₁₅C₂N₅O₂ 391/393 (calcd) 392/394 ([M+H]⁺ found).

### Example 12—Preparation of 2-(4,5-dichloro-6-oxopyridazin-1(6H)-yl)-N-(2,6-dichlorophenethyl)acetamide

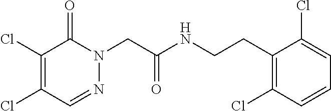 [SMILES: O=C(Cn1ncc(Cl)c(Cl)c1=O)NCCc1c(Cl)cccc1Cl]

The title compound was prepared according to the general procedure B as a white solid.

LC/MS (ESI) m/z for C₁₄H₁₁Cl₄N₃O₂ 393/395/397 (calcd) 394/396/398 ([M+H]⁺ found).

### Example 13—Preparation of rac-2-(4,5-dichloro-6-oxopyridazin-1(6H)-yl)-N-((1R,2S)-2-(1-phenyl-1H-pyrazol-4-yl)cyclopropyl)acetamide

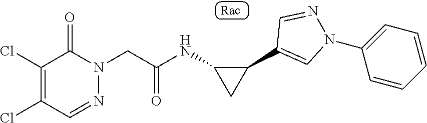 [SMILES: O=C(Cn1ncc(Cl)c(Cl)c1=O)N[C@H]1C[C@@H]1c1cnn(-c2ccccc2)c1]

The title compound was prepared according to the general procedure B as a white solid.

LC/MS (ESI) m/z for C₁₈H₁₅Cl₂N₅O₂ 403/405 (calcd) 404/406 ([M+H]⁺ found).

¹H NMR (400 MHz, CDCl₃) δ 7.86 (s, 1H), 7.78 (s, 1H), 7.62 (d, J=7.5 Hz, 2H), 7.53 (s, 1H), 7.42 (t, J=7.9 Hz, 2H), 7.24 (pseudo d, J=7.3 Hz, 1H), 6.33 (s, 1H), 4.81 (s, 2H), 2.84-2.77 (symmetrical m, 1H), 1.97 (ddd, J=9.7, 6.7, 3.3 Hz, 1H), 1.21-1.09 (m, 2H).

### Example 14—Preparation of 2-(4,5-dichloro-6-oxopyridazin-1(6H)-yl)-N-(1-phenyl-1H-pyrazol-4-yl)acetamide

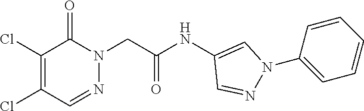 [SMILES: O=C(Cn1ncc(Cl)c(Cl)c1=O)Nc1cnn(-c2ccccc2)c1]

The title compound was prepared according to the general procedure B as a white solid.

LC/MS (ESI) m/z for C₁₅H₁₁C₁₂N₅O₂ 363/365 (calcd) 364/366 ([M+H]⁺ found).

### Example 15—Preparation of 2-(4,5-dichloro-6-oxopyridazin-1(6H)-yl)-N-((1-phenyl-1H-pyrazol-4-yl)methyl)acetamide

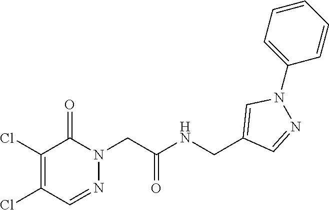 [SMILES: O=C(Cn1ncc(Cl)c(Cl)c1=O)NCc1cnn(-c2ccccc2)c1]

The title compound was prepared according to the general procedure B as a white solid.

LC/MS (ESI) m/z for C₁₆H₁₃Cl₂N₅O₂ 377/379 (calcd) 378/380 ([M+H]⁺ found).

### Example 16—Preparation of 4-chloro-2-(2-(2,4-dichlorophenyl)-2-oxoethyl) pyridazin-3(2H)-one

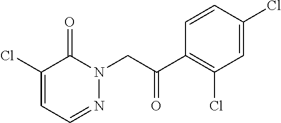 [SMILES: O=C(Cn1nccc(Cl)c1=O)c1ccc(Cl)cc1Cl]

The title compound was prepared according to the general procedure A as a white solid (16 mg, 99% yield).

LC/MS (ESI) m/z for C₁₂H₇Cl₃N₂O₂ 316/318/320 (calcd) 317/319/321 ([M+H]⁺ found).

¹H NMR (400 MHz, CDCl₃) δ 7.76 (d, J=4.4 Hz, 1H), 7.73 (d, J=8.4 Hz, 1H), 7.49 (d, J=2.0 Hz, 1H), 7.44 (d, J=4.5 Hz, 1H), 7.38 (dd, J=8.4, 1.9 Hz, 1H), 5.52 (s, 2H).

### Example 17—In Vitro Testing of Compounds

### Materials:

Compounds 1, 2, 7, 9 and 10 were purchased from Enamine. The other compounds tested were made in accordance with the Examples above.

### Cell Culture:

QBIHEK293A cells stably transfected with human (h) natriuretic peptide receptor A (hNPR-A) and hNPR-B were made as described in Bach et al., Naunyn Schmiedebergs Arch. Pharmacol. 2014; 387:5-14. Cells were grown in DMEM (Dulbecco's Modified Eagle's Medium, Gibco®, ThermoFischer Scientific) supplemented with 10% Fetal Bovine Serum (FBS), 100 U/ml penicillin, 0.1 mg/ml streptomycin, and 0.4 mg/ml G418 (geneticin) as selection antibiotic.

AlphaScreen Assay for cGMP:

The AlphaScreen assay for cGMP (Perkin Elmer) was performed as described in Bach et al. (see above). NPR-A and NPR-B expressing HEK293 cells were split the day before the experiment and harvested using an EDTA solution (Versene, Invitrogen, ThermoFischer Scientific). A final concentration of 6000 cells/well was re-suspended in stimulation buffer (5 mM HEPES in HBSS at pH 7.4, 0.1% BSA) with IBMX (0.7 mM final). Compounds were dissolved in stimulation buffer and added to wells in increasing concentrations. Cells were incubated with the indicated compounds 20 minutes before addition of agonists (human BNP, human CNP or human proBNP) in various concentrations. Cells were stimulated for 20 minutes with agonist before the reactions were stopped (i.e. cells lysed) by adding the AlphaScreen Acceptor bead mix (Acceptor beads-anti-cGMP antibody and 0.5% Tween-20 in 5 mM HEPES buffer at pH 7.4). After 1 hour incubation, the Donor bead mix (Donor beads, biotinylated cGMP, 0.5% Tween-20 in 5 mM HEPES buffer at pH 7.4) was added (40 μl total volume) and incubation continued for 2 hours. The luminescence signals were analyzed quantitatively by reading of the plates on the EnVision® Multilabel Plate Reader (Perkin Elmer) using AlphaScreen emission 570 nm filter. Experiments were performed either with 0.1 nM/2 nM of BNP/proBNP presence with increasing concentrations of the compound (Conc. resp. Compound+0.1 nM BNP/2 nM proBNP) or in the presence of 10 μM compound with increasing concentrations of BNP (Conc. resp. BNP/proBNP+10 μM compound) and concentration-response curves constructed giving −Log EC₅₀ (potency) and maximal response values (efficacy).

### Whole-Cell Binding Assays:

Binding assays were carried out in intact NPR-A expressing HEK293 cells as described in Dickey et al., J Biol Chem. 2009; 284: 19196-202.

### Vasorelaxation:

Male Sprague-Dawley (6-8 weeks old) rats were killed by cervical dislocation. The thoracic aorta was dissected and rings (˜4 mm length) mounted in organ baths containing physiological salt solution (PSS; composition in mM: NaCl 119, KCl 4.7, CaCl₂ 2.5, MgSO₄ 1.2, NaHCO₃ 25, KH₂PO₄ 1.2, and glucose 5.5), maintained at 37° C. and gassed with 5% CO₂ in O₂. Changes in isometric tension were measured in the tissues under a basal tension of 1 g. After equilibration, vessels were repeatedly contracted with KCl (48 mM) until responses were reproducible. A cumulative concentration-response curve was then constructed to the thromboxane receptor agonist 9,11-dideoxy-11α,9α-epoxymethano-prostaglandin F2α (U46619; 10 nm-1 μM). Arteries were then treated with the nitric oxide synthase (NOS) inhibitor L-NG-nitroarginine methyl ester (300 μM; to block the production of endogenous nitric oxide) and pre-contracted with an EC₈₀ concentration of U46619. Once a stable response was achieved, cumulative concentration-response curves were constructed to ANP (0.001-1 μM) in the absence or presence of Compound 1 or Compound 2 (all 10 μM, incubated for 30 min prior to the administration of ANP).

### MTT Assays:

The MTT (3-(4,5-dimethylthiazol-2-yl)-2,5-diphenyltetrazolium bromide) tetrazolium reduction assay was used to measure cytotoxicity of the compounds in vitro. Viable cells with active metabolism convert MTT into a purple colored formazan product with an absorbance maximum near 570 nm. HeLa cells were plated in 96-well plates in culture medium (DMEM (Dulbecco's Modified Eagle's Medium, Gibco®, ThermoFischer Scientific) supplemented with 10% Fetal Bovine Serum (FBS), 100 U/ml penicillin, 0.1 mg/ml streptomycin) the day before experiments. Cells were incubated for 24 or 48 h at 37° C. with increasing concentrations of each compound (in 1% DMSO). 1% DMSO and 20 mM H₂O₂ in culture medium were used as negative and positive control, respectively. MTT solution at a final concentration of 0.45 mg/ml was added to the wells and cells incubated for 1 to 4 hours at 37° C. Solubilization solution (40% (vol/vol) dimethylformamide (DMF), 2% (vol/vol) glacial acetic acid and 16% (wt/vol) sodium dodecyl sulfate (SDS)) was added to each well to dissolve formazan crystals. Absorbance was measured at 570 nm.

### Results:

Results on cGMP production, binding affinity data and effect on vasorelaxation are provided in Tables 1 to 4 below.

Results from the MTT assays are shown in FIG. 2. The monochloro-analogues (compounds 2 and 24) exhibited lower toxicity than the corresponding dichloro compounds (compounds 3 and 11).

Compound 24 did not show any cytotoxic effects on parameters such as cell count, nuclear size, DNA structure, cell membrane permeability, mitochondrial mass, mitochondrial membrane potential and cytochrome c (cytotoxicity screening was performed by Cyprotex Discovery Ltd. (Cheshire, UK)) at concentrations below 15 μM (>70-80-fold higher concentration than EC₅₀ of compound 24 at the target). Compound 24 was tested for selectivity on agonistic/antagonistic/inhibitory/blocker effect on 78 different targets (Safety pharmacology panel (SafetyScan E/IC50 ELECT (Discover X/Eurofins)). Of the 78 targets, compound 24 showed minor activity against dopamine D₁-receptor, β₁- and β₂-adrenergic receptor with IC₅₀ values above 6 μM.

TABLE 1
BNP-mediated NPR-A activity (cGMP production) of compounds

| | | Conc. response of | | | | | Conc. response of BNP + 10 μM | | | | |
|---|---|---|---|---|---|---|---|---|---|---|---|
| | | compound + 0.1 nM BNP^{a} | | | | | compound^{b} | | | | |
| Com- | | | -LogEC₅₀ | | Max | | | | | Max | |
| pound | | | (com- | | Re- | | | -LogEC₅₀ | | Re- | |
| No. | Structure | n | pound) | SEM^{c} | sponse | SEM^{c} | n | (BNP) | SEM^{c} | sponse | SEM^{c} |
| BNP | | | | | | | 20 | 9.1 | 0.2 | 100.0 | |
| 1 | 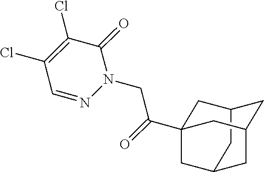 [SMILES: O=C(Cn1ncc(Cl)c(Cl)c1=O)C12CC3CC(CC(C3)C1)C2] | 3 | 5.6 | 0.3 | 53.1 | 4.5 | 2 | 9.5 | 0.1 | 144.4 | 14.4 |
| 2 | 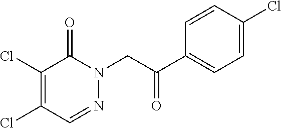 [SMILES: O=C(Cn1ncc(Cl)c(Cl)c1=O)c1ccc(Cl)cc1] | 18 | 6.6 | 0.1 | 45.3 | 3.0 | 20 | 9.5 | 0.2 | 146.2 | 5.5 |
| 3 | 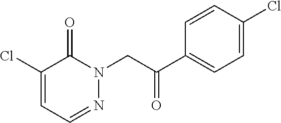 [SMILES: O=C(Cn1nccc(Cl)c1=O)c1ccc(Cl)cc1] | 5 | 4.9 | 0.1 | 48.6 | 3.5 | 6 | 9.3 | 0.1 | 102.9 | 3.2 |
| 4 | 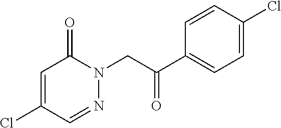 [SMILES: O=C(Cn1ncc(Cl)cc1=O)c1ccc(Cl)cc1] | 5 | 5.3 | 0.1 | 35.2 | 3.8 | 5 | 9.3 | 0.2 | 101.7 | 3.6 |
| 5 | 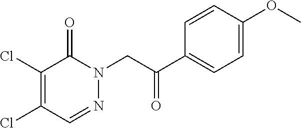 [SMILES: COc1ccc(C(=O)Cn2ncc(Cl)c(Cl)c2=O)cc1] | 3 | 5.4 | 0.2 | 38.6 | 0.8 | 3 | 9.3 | 0.2 | 126.9 | 8.4 |
| 6 | 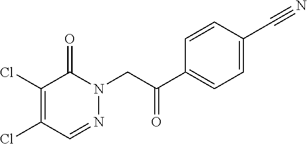 [SMILES: N#Cc1ccc(C(=O)Cn2ncc(Cl)c(Cl)c2=O)cc1] | 3 | 5.6 | 0.2 | 44.1 | 2.7 | 3 | 9.5 | 0.0 | 145.5 | 5.6 |
| 7 | 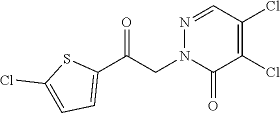 [SMILES: O=C(Cn1ncc(Cl)c(Cl)c1=O)c1ccc(Cl)s1] | 2 | 6.6 | 0.5 | 33.4 | 2.9 | 4 | 9.2 | 0.2 | 101.8 | 8.9 |
| 8 | 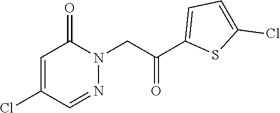 [SMILES: O=C(Cn1ncc(Cl)cc1=O)c1ccc(Cl)s1] | 3 | 5.8 | 0.3 | 27.0 | 2.2 | 4 | 9.3 | 0.1 | 131.7 | 22.8 |
| 9 | 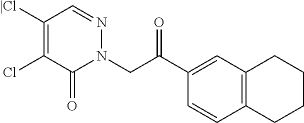 | 1 | 5.9 | | 27.7 | | 1 | 9.4 | | 123.3 | |
| 10 | 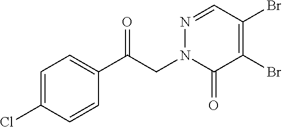 [SMILES: O=C(Cn1ncc(Br)c(Br)c1=O)c1ccc(Cl)cc1] | 3 | 7.1 | 0.2 | 37.0 | 3.1 | 4 | 9.3 | 0.2 | 179.0 | 17.6 |
| 11 | 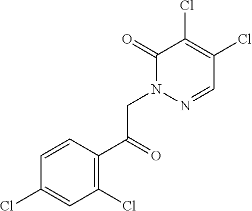 [SMILES: O=C(Cn1ncc(Cl)c(Cl)c1=O)c1ccc(Cl)cc1Cl] | 4 | 6.7 | 0.1 | 42.5 | 3.0 | 4 | 9.5 | 0.1 | 148.5 | 4.6 |
| 12 | 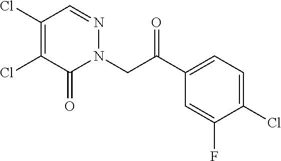 [SMILES: O=C(Cn1ncc(Cl)c(Cl)c1=O)c1ccc(Cl)c(F)c1] | 2 | 6.4 | 0.0 | 44.3 | 1.2 | 2 | 9.4 | 0.1 | 126.4 | 7.2 |
| 13 | 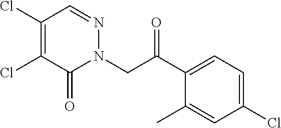 [SMILES: Cc1cc(Cl)ccc1C(=O)Cn1ncc(Cl)c(Cl)c1=O] | 2 | 7.0 | 0.2 | 41.0 | 2.6 | 2 | 9.6 | 0.3 | 107.7 | 15.8 |
| 14 | 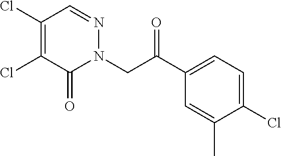 [SMILES: Cc1cc(C(=O)Cn2ncc(Cl)c(Cl)c2=O)ccc1Cl] | 2 | 5.6 | 0.0 | 45.4 | 4.5 | 2 | 9.5 | 0.1 | 108.3 | 3.5 |
| 15 | 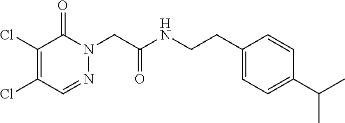 [SMILES: CC(C)c1ccc(CCNC(=O)Cn2ncc(Cl)c(Cl)c2=O)cc1] | 2 | 6.1 | 0.4 | 27.8 | 9.1 | 2 | 9.2 | 0.1 | 169.6 | 18.0 |
| 16 | 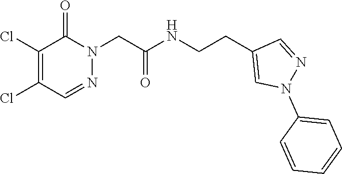 [SMILES: O=C(Cn1ncc(Cl)c(Cl)c1=O)NCCc1cnn(-c2ccccc2)c1] | 2 | 6.1 | 0.0 | 41.0 | 2.6 | 2 | 9.3 | 0.1 | 124.3 | 11.7 |
| 17 | 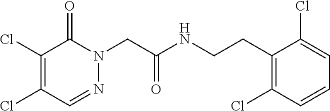 [SMILES: O=C(Cn1ncc(Cl)c(Cl)c1=O)NCCc1c(Cl)cccc1Cl] | 2 | 6.1 | 2.0 | 39.4 | 7.7 | 2 | 9.1 | 0.2 | 110.7 | 0.7 |
| 18 | 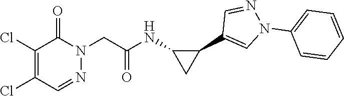 [SMILES: O=C(Cn1ncc(Cl)c(Cl)c1=O)N[C@H]1C[C@@H]1c1cnn(-c2ccccc2)c1] | 2 | 6.7 | 0.2 | 30.7 | 16.1 | 2 | 9.3 | 0.0 | 141.5 | 21.3 |
| 19 | 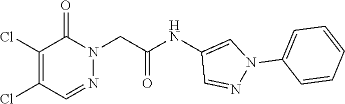 [SMILES: O=C(Cn1ncc(Cl)c(Cl)c1=O)Nc1cnn(-c2ccccc2)c1] | 2 | 6.0 | 0.1 | 45.7 | 0.1 | 2 | 9.3 | 0.0 | 125.4 | 3.2 |
| 20 | 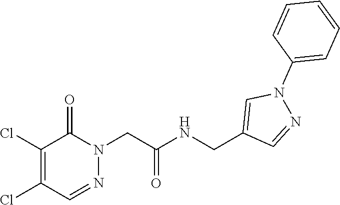 [SMILES: O=C(Cn1ncc(Cl)c(Cl)c1=O)NCc1cnn(-c2ccccc2)c1] | 2 | 5.2 | 0.3 | 49.4 | 3.7 | 2 | 9.2 | 134.5 | 2.1 | |
| 24 | 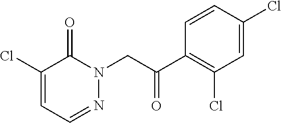 [SMILES: O=C(Cn1nccc(Cl)c1=O)c1ccc(Cl)cc1Cl] | 4 | 6.7 | 0.2 | 37.8 | 1.6 | 4 | 9.8 | 0.04 | 122.7 | 1.3 |

^{a}-LogEC₅₀ (compound) and max response is based on construction of concentration-response curves for the compounds in the presence of 0.1 nM BNP in NPR-
A-expressing HEK293 cells. Max response is defined as the percentage (%) of maximum BNP-mediated cGMP production.
^{b}-LogEC₅₀ (BNP) and max response are based on construction of concentration-response curves for BNP in the absence (BNP) or presenceof 10 μM compound.
The max response is the maximum BNP-mediated cGMP production where BNP alone is defined as 100%.
^{c}If n = 2, standard deviation is given as half-maximum range.

TABLE 2
proBNP-mediated NPR-A activity of compounds

| | | Conc. response of compound + 2 nM | | | | | Conc. response of proBNP + 10 μM | | | | |
|---|---|---|---|---|---|---|---|---|---|---|---|
| | | proBNP^{a} | | | | | compound^{b} | | | | |
| Compound | | | -LOGEC₅₀ | | Max | | | -LogEC₅₀ | | Max | |
| No. | Structure | n | (compound) | SEM | response | SEM | n | (proBNP) | SEM | Response | SEM |
| proBNP | | | | | | | 4 | 8.4 | 0.1 | 100.0 | |
| 1 | 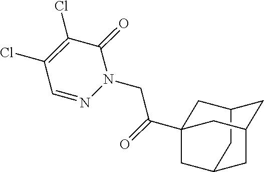 [SMILES: O=C(Cn1ncc(Cl)c(Cl)c1=O)C12CC3CC(CC(C3)C1)C2] | | | | | | 4 | 8.1 | 0.1 | 186.0 | 5.0 |
| 2 | 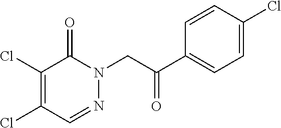 [SMILES: O=C(Cn1ncc(Cl)c(Cl)c1=O)c1ccc(Cl)cc1] | 5 | 6.0 | 0.2 | 99.0 | 6.0 | 4 | 8.7 | 0.1 | 189.0 | 6.0 |

^{a}-LogEC₅₀ (compound) and max response is based on construction of concentration-response curves for the compounds in the presence of 2 nM
proBNP in NPR-A-expressing HEK293 cells. Max response is defined as the percentage (%) of maximum proBNP-mediated cGMP production.
^{b}-LogEC₅₀ (proBNP) and max response are based on construction of concentration-response curves for proBNP in the absence (proBNP) or
presence of 10 μM compound. The max response is the maximum proBNP-mediated cGMP production where proBNP alone is defined as 100%.

TABLE 3
Binding affinity data

| | | | | | | | Saturation binding (conc. response | | | | |
|---|---|---|---|---|---|---|---|---|---|---|---|
| | | Conc. response compound + 50 pM ¹²⁵I-ANP^{a} | | | | | ¹²⁵I-ANP + 10 μM compound)^{b} | | | | |
| Compound | | | -LogEC₅₀ | | Max | | | -Log K_{d} | | Fold change | |
| No. | Structure | n | (compound) | SEM | Response | SEM | n | (¹²⁵I-ANP) | SEM | in Bmax | SEM |
| ¹²⁵I-ANP | | | | | 100.0 | | | 8.9 | 0.1 | 0 | |
| 2 | 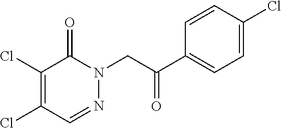 [SMILES: O=C(Cn1ncc(Cl)c(Cl)c1=O)c1ccc(Cl)cc1] | 3 | 8.7 | 0.1 | 173.0 | 7.0 | 4 | 9.0 | 0.1 | 2.3 | 0.1 |

^{a}-LogEC₅₀ and max response are based on construction of competition binding curves using stably expressing NPR-A HEK293 cells with increasing
concentrations of compound 2 in presence of 50 pM ¹²⁵I-ANP where the response of 50 pM ¹²⁵I-ANP is defined as 100%.
^{b}-Log K_{d} and fold change in Bₘₐₓ are based on saturation binding analysis of ¹²⁵I-ANP using stably NPR-A expressing HEK293 cells. Cells were
incubated with increasing concentrations of ¹²⁵I-ANP for 1 hour at 4 °C. in the presence or absence of 1 μM ANP to determine specific and non-specific
binding and in the absence of 10 μM compound 2.

TABLE 4
Activity on vasorelaxation

| Compound | | Conc. response of ANP + 10 μM compound^{b} | | |
|---|---|---|---|---|
| No. | Structure | n | -LogEC₅₀ (ANP) | SEM |
| ANP | | 6 | 8.3 | 0.1 |
| 1 | 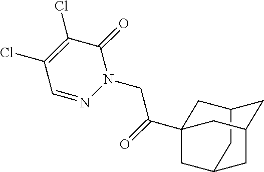 [SMILES: O=C(Cn1ncc(Cl)c(Cl)c1=O)C12CC3CC(CC(C3)C1)C2] | 6 | 8.7 | 0.1 |
| 2 | 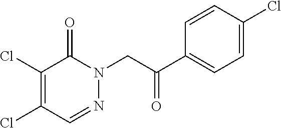 [SMILES: O=C(Cn1ncc(Cl)c(Cl)c1=O)c1ccc(Cl)cc1] | 6 | 8.9 | 0.1 |

^{b}-LogEC₅₀ (ANP) is based on construction of concentration-response curve for ANP-mediated relaxation of
phenylephrine-induced tone in rat aortic rings in the presence of L-NAME and in the absence (ANP) or presence
of 10 μM compound.

### Example 18—Injection Formulation

Sulfobutylether-β-cyclodextrin sodium salt (30 g, BioSynth CarboSynth) was dissolved in sterile water (to 100 ml). Compound 24 (2.54 mg) was dissolved in 10 ml of the resulting solution by stirring and heating to approx. 100° C. The solution was sterile filtered and aseptically filled into sterile 2 ml injection vials.

1 ml solution comprised 0.254 mg of compound 24. The concentration of the compound was 800 μM.

The solution is suitable for administration intravenously, intramuscularly or subcutaneously.

### Example 19—Gelatin Capsule

Hard gelatin capsules containing compound 24 can be prepared as described below:

| | | |
|---|---|---|
| Compound 24 | 20 | g |
| Magnesium stearate | 1 | g |
| Mannitol | 50 | g |

| | |
|---|---|
| Microcrystalline cellulose | q.s to 500 g |

These components are volumetrically mixed in a mixer and filled into 1000 hard gelatin capsules size 0. Each capsule comprises 15 mg of the active compound 24. The capsules are packaged in standard capsule boxes (100 capsules per box), labelled and packed with a package insert.

1. A compound of formula (I), stereoisomer, or pharmaceutically acceptable salt thereof for use in the treatment or prevention of hypertension: 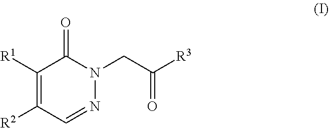
wherein:
R¹ and R² are independently selected from:
H
halogen (e.g. F, Cl or Br),
C₁₋₆ alkyl (e.g. C₁₋₃ alkyl),
C₁₋₆ haloalkyl (e.g. C₁₋₃ haloalkyl), and
CN,
provided that at least one of R¹ and R² is other than —H;
R³ is a group of the formula: 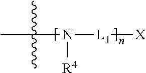
in which n is 0 or 1;
R⁴ is H or C₁₋₃ alkyl (e.g. —CH₃);
L₁ is a linking group selected from:
a bond,
—(CH₂)ₚ— in which p is an integer from 1 to 3, preferably 1 or 2, and
C₃₋₆ cycloalkylene (e.g. C₃₋₄ cycloalkylene);
and
X is selected from:
an aryl group optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), and —CN;
a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and optionally substituted phenyl (e.g. unsubstituted phenyl); and
a cycloalkyl group (e.g. a tricyclic cycloalkyl group) optionally substituted by one or more substituents selected from C₁₋₃ alkyl (e.g. —CH₃), C₁₋₃ haloalkyl (e.g. —CF₃), and halogen (e.g. F, Cl or Br).

2. A compound for use as claimed in claim 1, wherein said hypertension is resistant hypertension.

3. A compound for use as claimed in claim 1 in the treatment or prevention of pulmonary hypertension, hypertension in pregnancy, gestational hypertension or pregnancy-induced hypertension.

4. A compound for use as claimed in any one of the preceding claims, wherein in formula (I), R¹ and R² are independently selected from H and halogen, preferably from H, C and Br.

5. A compound for use as claimed in claim 4, wherein in formula (I), R¹ and R² are both halogen, preferably both Cl.

6. A compound for use as claimed in claim 4, wherein in formula (I), R¹ is halogen (e.g. C) and R² is H.

7. A compound for use as claimed in any one of claims 1 to 6 which is a compound of formula (Ia), a stereoisomer, or a pharmaceutically acceptable salt thereof: 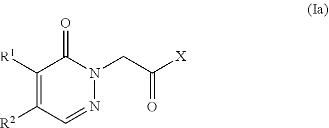
wherein:
R¹ and R² are as defined in any one of claims 1 and 4 to 6; and
X is selected from:
an aryl group optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and —CN;
a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and optionally substituted phenyl (e.g. unsubstituted phenyl); and
a cycloalkyl group (e.g. a tricyclic cycloalkyl group) optionally substituted by one or more substituents selected from C₁₋₃ alkyl (e.g. —CH₃), C₁₋₃ haloalkyl (e.g. —CF₃), and halogen (e.g. F, C or Br).

8. A compound for use as claimed in any one of claims 1 to 6 which is a compound of formula (Ib), a stereoisomer, or a pharmaceutically acceptable salt thereof: 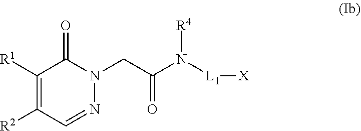
wherein:
R¹, R², R⁴ and L₁ are as defined in any one of claims 1 and 4 to 6; and
X is selected from:
an aryl group optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), and —CN; and
a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and optionally substituted phenyl (e.g. unsubstituted phenyl).

9. A compound for use as claimed in any one of claims 1 to 6 which is a compound of formula (Ic), a stereoisomer, or a pharmaceutically acceptable salt thereof: 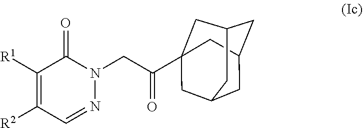
wherein:
R¹ and R² are as defined in any one of claims 1 and 4 to 6.

10. A compound for use as claimed in any one of claims 1 to 6 which is a compound of formula (Id), a stereoisomer, or a pharmaceutically acceptable salt thereof: 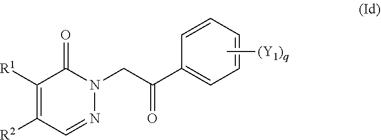
wherein:
R¹ and R² are as defined in any one of claims 1 and 4 to 6;
each Y₁ is independently selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), and —CN; and
q is an integer from 0 to 3, preferably 1 or 2.

11. A compound for use as claimed in any one of claims 1 to 6 which is a compound of formula (Ie), a stereoisomer, or a pharmaceutically acceptable salt thereof: 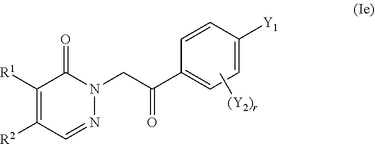
wherein:
R¹ and R² are as defined in any one of claims 1 and 4 to 6;
Y₁ is as defined in claim 10, preferably C₁₋₃ alkoxy (e.g. —OCH₃), C₁₋₃ haloalkyl (e.g. —CF₃), halogen (e.g. F, C or Br), or —CN;
each Y₂ is independently selected from C₁₋₃ alkyl (e.g. —CH₃), C₁₋₃ alkoxy (e.g. —OCH₃), C₁₋₃ haloalkyl (e.g. —CF₃), halogen (e.g. F, Cl or Br), or —CN, preferably halogen (e.g. F, Cl or Br); and
r is an integer from 0 to 2, preferably 0 or 1.

12. A compound for use as claimed in any one of claims 1 to 6 which is a compound of formula (If), a stereoisomer, or a pharmaceutically acceptable salt thereof: 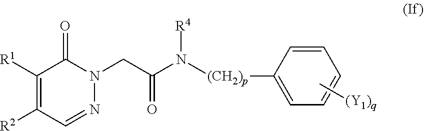
wherein:
R¹, R², R⁴ and p are as defined in any one of claims 1 and 4 to 6;
each Y₁ is independently selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), and —CN; and
q is an integer from 0 to 3, preferably 1 or 2.

13. A compound for use as claimed in any one of claims 1 to 6 which is a compound of formula (Ig), a stereoisomer, or a pharmaceutically acceptable salt thereof: 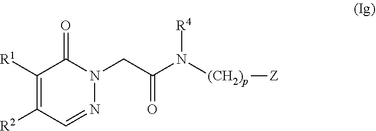
wherein:
R¹, R², R⁴ and p are as defined in any one of claims 1 and 4 to 6; and
Z is a 5 or 6-membered heterocyclic ring optionally substituted by one or more substituents selected from C₁₋₃ alkyl (e.g. —CH₃), C₁₋₃ alkoxy (e.g. —OCH₃), C₁₋₃ haloalkyl (e.g. —CF₃), halogen (e.g. F, C or Br), and optionally substituted phenyl (e.g. unsubstituted phenyl).

14. A compound for use as claimed in any one of claims 1 to 3 selected from any of the following compounds, their stereoisomers, and their pharmaceutically acceptable salts:

| Compound No. | Structure |
|---|---|
| 1 | 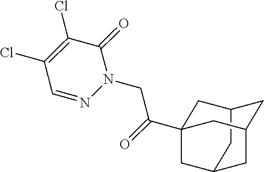 [SMILES: O=C(Cn1ncc(Cl)c(Cl)c1=O)C12CC3CC(CC(C3)C1)C2] |
| 2 | 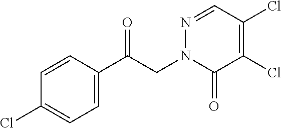 [SMILES: O=C(Cn1ncc(Cl)c(Cl)c1=O)c1ccc(Cl)cc1] |
| 3 | 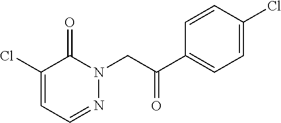 [SMILES: O=C(Cn1nccc(Cl)c1=O)c1ccc(Cl)cc1] |
| 4 | 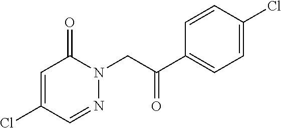 [SMILES: O=C(Cn1ncc(Cl)cc1=O)c1ccc(Cl)cc1] |
| 5 | 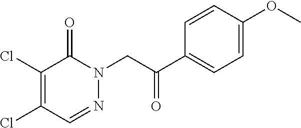 [SMILES: COc1ccc(C(=O)Cn2ncc(Cl)c(Cl)c2=O)cc1] |
| 6 | 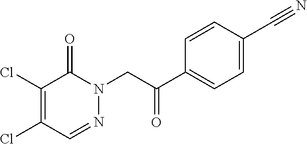 [SMILES: N#Cc1ccc(C(=O)Cn2ncc(Cl)c(Cl)c2=O)cc1] |
| 7 | 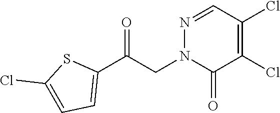 [SMILES: O=C(Cn1ncc(Cl)c(Cl)c1=O)c1ccc(Cl)s1] |
| 8 | 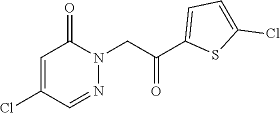 [SMILES: O=C(Cn1ncc(Cl)cc1=O)c1ccc(Cl)s1] |
| 9 | 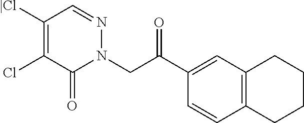 |
| 10 | 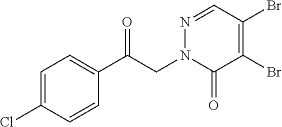 [SMILES: O=C(Cn1ncc(Br)c(Br)c1=O)c1ccc(Cl)cc1] |
| 11 | 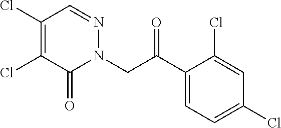 [SMILES: O=C(Cn1ncc(Cl)c(Cl)c1=O)c1ccc(Cl)cc1Cl] |
| 12 | 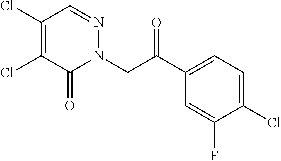 [SMILES: O=C(Cn1ncc(Cl)c(Cl)c1=O)c1ccc(Cl)c(F)c1] |
| 13 | 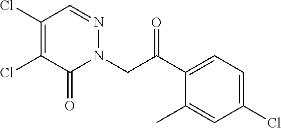 [SMILES: Cc1cc(Cl)ccc1C(=O)Cn1ncc(Cl)c(Cl)c1=O] |
| 14 | 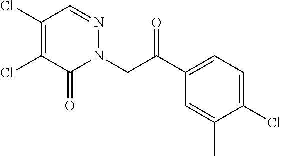 [SMILES: Cc1cc(C(=O)Cn2ncc(Cl)c(Cl)c2=O)ccc1Cl] |
| 15 | 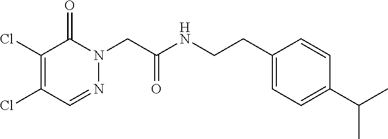 [SMILES: CC(C)c1ccc(CCNC(=O)Cn2ncc(Cl)c(Cl)c2=O)cc1] |
| 16 | 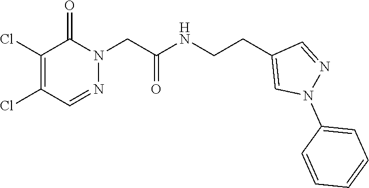 [SMILES: O=C(Cn1ncc(Cl)c(Cl)c1=O)NCCc1cnn(-c2ccccc2)c1] |
| 17 | 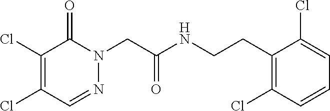 [SMILES: O=C(Cn1ncc(Cl)c(Cl)c1=O)NCCc1c(Cl)cccc1Cl] |
| 18 | 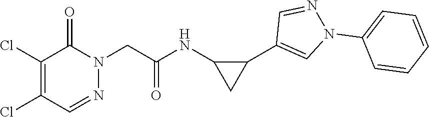 [SMILES: O=C(Cn1ncc(Cl)c(Cl)c1=O)NC1CC1c1cnn(-c2ccccc2)c1] |
| 19 | 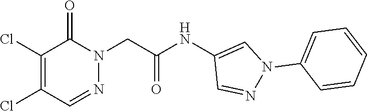 [SMILES: O=C(Cn1ncc(Cl)c(Cl)c1=O)Nc1cnn(-c2ccccc2)c1] |
| 20 | 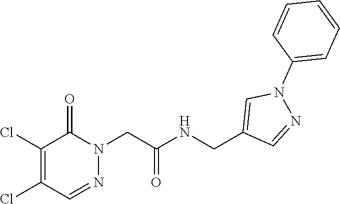 [SMILES: O=C(Cn1ncc(Cl)c(Cl)c1=O)NCc1cnn(-c2ccccc2)c1] |
| 21 | 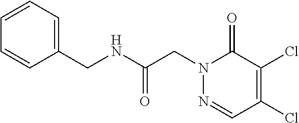 [SMILES: O=C(Cn1ncc(Cl)c(Cl)c1=O)NCc1ccccc1] |
| 22 | 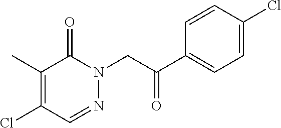 [SMILES: Cc1c(Cl)cnn(CC(=O)c2ccc(Cl)cc2)c1=O] |
| 23 | 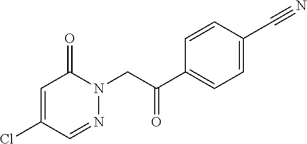 [SMILES: N#Cc1ccc(C(=O)Cn2ncc(Cl)cc2=O)cc1] |
| 24 | 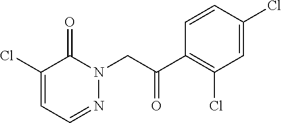 [SMILES: O=C(Cn1nccc(Cl)c1=O)c1ccc(Cl)cc1Cl] |

15. A pharmaceutical composition comprising a compound, stereoisomer, or pharmaceutically acceptable salt thereof as defined in any one of claims 1 and 4 to 14, together with one or more pharmaceutically acceptable carriers, excipients or diluents, and which further comprises an additional anti-hypertensive agent.

16. A pharmaceutical composition as claimed in claim 15 which is adapted for oral administration.

17. A compound, stereoisomer, or pharmaceutically acceptable salt thereof for use as claimed in any one of claims 1 to 14, wherein said compound, stereoisomer, or pharmaceutically acceptable salt is administered in combination with (e.g. simultaneously, separately or sequentially) an additional anti-hypertensive agent.

18. A compound, stereoisomer, or pharmaceutically acceptable salt thereof for use as claimed in claim 17, wherein said anti-hypertensive agent is an inhibitor of the renin-angiotensin-aldosterone system, a thiazide diuretic, a calcium channel blocker, or a beta-blocker.

19. A compound, stereoisomer, or pharmaceutically acceptable salt thereof for use as claimed in claim 17, wherein said anti-hypertensive agent is selected from benazepril, zofenopril, perindopril, trandolapril, captopril, enalapril, lisinopril, ramipril, losartan, telmisartan, irbesartan, candesartan, olmesartan, valsartan, saprisartan, hydrochlorothiazide, bendroflumethiazide, chlorthalidone, amlodipine, felodipine, isradipine, nifedipine, nimodipine, lerkanidipine, verapamil, dilthiazem, metoprolol, propranolol, timolol, atenolol, bisoprolol, esmolol, carvedilol, nebivolol, spironolactone, eplerenone, prazosine, tamsulozine, sacubitril, and sacubritril-valsartan.

20. A package comprising: (i) a compound, stereoisomer or pharmaceutically acceptable salt as defined in any one of claims 1 and 4 to 14, or a pharmaceutical composition comprising said compound, stereoisomer or pharmaceutically acceptable salt; and (ii) printed instructions and/or a label relating to the use of (i) in the treatment of any of the conditions or disorders defined in any one of claims 1 to 3, for example in the treatment or prevention of resistant hypertension.

21. A compound of general formula (II), or a stereoisomer, or pharmaceutically acceptable salt thereof: 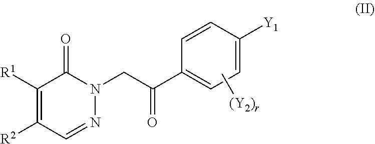
wherein:
R¹ and R² are as defined in any one of claims 1 and 4 to 6;
Y₁ is as defined in claim 10, preferably C₁₋₃ alkoxy (e.g. —OCH₃), C₁₋₃ haloalkyl, halogen (e.g. F, Cl or Br), or —CN;
each Y₂ is independently selected from C₁₋₃ alkyl (e.g. —CH₃), C₁₋₃ alkoxy (e.g. —OCH₃), C₁₋₃ haloalkyl, halogen (e.g. F, Cl or Br), or —CN, preferably halogen (e.g. F, C or Br); and
r is an integer from 0 to 2, preferably 0 or 1;
with the proviso that the compound is other than:
4-chloro-2-[2-(2,4-dichlorophenyl)-2-oxo-ethyl]pyridazin-3-one;
4,5-dichloro-2-[2-(4-chlorophenyl)-2-oxo-ethyl]pyridazin-3-one;
4,5-dichloro-2-[2-(4-methoxyphenyl)-2-oxo-ethyl]pyridazin-3-one; or
4,5-dichloro-2-[2-(4-methylphenyl)-2-oxo-ethyl]pyridazin-3-one.

22. A compound as claimed in claim 21 having the general formula (IIa), or a stereoisomer, or pharmaceutically acceptable salt thereof: 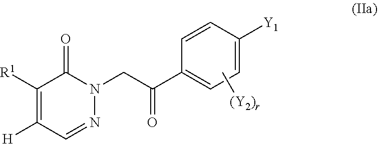
wherein:
R¹ is halogen (e.g. F, Cl or Br), C₁₋₆ alkyl (e.g. C₁₋₃ alkyl), C₁₋₆ haloalkyl (e.g. C₁₋₃ haloalkyl) or —CN, preferably halogen (e.g. F, Cl or Br), e.g. Cl;
Y₁ is as defined in claim 10, preferably C₁₋₃ alkoxy (e.g. —OCH₃), C₁₋₃ haloalkyl, halogen (e.g. F, Cl or Br), or —CN;
each Y₂ is independently selected from C₁₋₃ alkyl (e.g. —CH₃), C₁₋₃ alkoxy (e.g. —OCH₃), C₁₋₃ haloalkyl, halogen (e.g. F, C or Br), or —CN, preferably halogen (e.g. F, C or Br); and
r is an integer from 0 to 2, preferably 0 or 1.

23. A compound as claimed in claim 21 which is a compound denoted by the No. 3, 4, 6, 11, 12, 13, 14 or 24 in claim 14, or a stereoisomer or pharmaceutically acceptable salt thereof.

24. A compound as claimed in claim 23 which is a compound denoted by the No. 24, or a stereoisomer of pharmaceutically acceptable salt thereof.
